SEQUENCE LISTING (7 sequences)

SEQ ID NO: 1 - DNA, 954 bp, Triticum spp
  misc_feature (243)..(243): n is a, c, g, or t
atggcgcgcctcctcgttctcgccgtcacggccacggttctcatggtgcgaaagagctagctagtagagccggccatgcatggcacagatacctatcatacacgactgaattttgtcttgtcaaacatttcatcggtgcgttttttttgtctctccaggctcaatccgggcagccggcgtctgctgcgcgccgccctgcccgacgcccccatgccggacgccatcctcgagctcctgccccantttgatcaccacgcatcaacggaacagggtactgattttgtggccatcttccgatggaaggccgtctctctctagctcacccacgtgccttgctgcatgaatgcagagaaagacactccggaaggcgcggtcgaggacgtggaggacaaggacccgccgccgcccatgaacttcaactacgactacgatgacgccttgccccggagcgaaaccaccagcgccccctcccccgacgtcctactgaaccgcgccgccgtcgtccgcaacgtcgccacgccgtcgtcggcggtgttcttcctcgaggacgcggtgcgcgtccgggagagcctgcccttccacaggatccatcgggccaccggcgctgccgaggcgtcggcagaacagccgctggagctgtacactgtgcactccgtgagggcggtcgaggggtccaatttcatcctgtgccggggtgaagccggcgaaggggccgtgtacgggtgccgcgcaaccggcccggcgagggcctacgtcctggccctggccggcgagcgcggggacgtgacgatgaccgcggttgccgtgtgccgcaccgacgcatcccgatgggacccggagcacgccgccttccggctcctgggcgtgaagcccggcggcgcggcggtctgccacgcggtgcgggacgcgcagctcctgccggccatgaacgggaagagccccgtcgccaactaa

SEQ ID NO: 2 - DNA, 615 bp, Artificial Sequence
  Nucleotide sequence C-Ph1 CDS
atgaatgcagagaaagacactccggaaggcgcggtcgaggacgtggaggacaaggacccgccgccgcccatgaacttcaactacgactacgatgacgccttgccccggagcgaaaccaccagcgccccctcccccgacgtcctactgaaccgcgccgccgtcgtccgcaacgtcgccacgccgtcgtcggcggtgttcttcctcgaggacgcggtgcgcgtccgggagagcctgcccttccacaggatccatcgggccaccggcgctgccgaggcgtcggcagaacagccgctggagctgtacactgtgcactccgtgagggcggtcgaggggtccaatttcatcctgtgccggggtgaagccggcgaaggggccgtgtacgggtgccgcgcaaccggcccggcgagggcctacgtcctggccctggccggcgagcgcggggacgtgacgatgaccgcggttgccgtgtgccgcaccgacgcatcccgatgggacccggagcacgccgccttccggctcctgggcgtgaagcccggcggcgcggcggtctgccacgcggtgcgggacgcgcagctcctgccggccatgaacgggaagagccccgtcgccaactaa

SEQ ID NO: 3 - DNA, 666 bp, Artificial Sequence
  Nucleotide sequence C-Ph1-Alt CDS
atgccggacgccatcctcgagctcctgccccagtttgatcaccacgcatcaacggaacaggagaaagacactccggaaggcgcggtcgaggacgtggaggacaaggacccgccgccgcccatgaacttcaactacgactacgatgacgccttgccccggagcgaaaccaccagcgccccctcccccgacgtcctactgaaccgcgccgccgtcgtccgcaacgtcgccacgccgtcgtcggcggtgttcttcctcgaggacgcggtgcgcgtccgggagagcctgcccttccacaggatccatcgggccaccggcgctgccgaggcgtcggcagaacagccgctggagctgtacactgtgcactccgtgagggcggtcgaggggtccaatttcatcctgtgccggggtgaagccggcgaaggggccgtgtacgggtgccgcgcaaccggcccggcgagggcctacgtcctggccctggccggcgagcgcggggacgtgacgatgaccgcggttgccgtgtgccgcaccgacgcatcccgatgggacccggagcacgccgccttccggctcctgggcgtgaagcccggcggcgcggcggtctgccacgcggtgcgggacgcgcagctcctgccggccatgaacgggaagagccccgtcgccaactaa

SEQ ID NO: 4 - DNA, 200 bp, Artificial Sequence
  Nucleotide sequence C-Ph1-RNAi sequence
cgtcctactaaaccgcgctgccgtcgtcacgccgtcgtcgacggtgttcttcctcgaggacgcggtgcgcgtcggggagagcctgcccttccacaggatccatcgggccaccgccgccgccgaggcgtcggcagagcagccgctggagctgtacaccgtccgctccgtgagggcggtcgaggggtccagtttcgtcctgt

SEQ ID NO: 5 - DNA, 98 bp, Artificial Sequence
  Nucleotide sequence C-Ph1-VIGS-as
cggtcgaggccgtggaggacaaggacccgccgccgcccatgaacttcaactacgactacgacgacgccttgccccggagcgaagccaccagcgccccc

SEQ ID NO: 6 - DNA, 45 bp, Artificial Sequence
  Nucleotide sequence C-Ph1-VIGS-hp1
cgctgccgtcgtcacgccgtcgtcgacggtgttcttcctcgagga

SEQ ID NO: 7 - DNA, 40 bp, Artificial Sequence
  Nucleotide sequence C-Ph1-VIGS-hp2
cgtgagggcggtcgaggggtccagtttcgtcctgtgccgg

(19) United States
(12) Patent Application Publication
(10) Pub. No.: US 2016/0251668 A1
(43) Pub. Date: Sep. 1, 2016

(54) COMPOSITIONS AND USES OF METAPHASE I SPECIFIC GENE SILENCING FOR EFFICIENT TRANSFER AND GENE MANIPULATION

(71) Applicant: Washington State University, Pullman, WA (US)

(72) Inventors: Kulvinder S. GILL, Pullman, WA (US); Ramanjot K. BHULLAR, Pullman, WA (US); Ragupathi NAGARAJAN, Pullman, WA (US)

(21) Appl. No.: 14/856,150

(22) Filed: Sep. 16, 2015

Related U.S. Application Data
(60) Provisional application No. 62/050,764, filed on Sep. 16, 2014.

(51) Int. Cl. C12N 15/82

(52) U.S. Cl. CPC C12N 15/8218

(57) ABSTRACT

The present disclosure provides improved compositions and methods to induce homoeologous pairing by post transcriptional silencing of C-Ph1 gene expression in a plant and identifies gene(s) responsible for the Ph1 gene-like function. The disclosure also provides hairpin and antisense vector constructs for reducing gene expression of a C-Ph1 gene in a plant.

### CROSS-REFERENCE TO RELATED APPLICATIONS

This application claims the benefit under 35 USC §119(e) of U.S. Provisional Application Ser. No. 62/050,764, filed on Sep. 16, 2014, the entire disclosure of which is incorporated herein by reference.

### BACKGROUND AND SUMMARY OF THE INVENTION

For consistent improvement in the yield and quality of plant species, a continual search for new genes and gene combinations is highly desired. Furthermore, researchers are continually searching for efficient methods of transferring and manipulating genes without undesirable carry-over genes. For example, wild relatives of wheat (such as rye) contain many agronomically important genes that could potentially be transferred to wheat.

Maintaining diploid like pairing behavior is essential for higher plants such as polyploids to establish as a new species. For example, the Pairing homoeologous 1 (Ph1) gene, which regulates such behavior in polyploid wheat, was identified in 1958 but its molecular function remained elusive. Because of the Ph1 gene, large alien segments carrying undesirable genes along with desirable genes are often transferred with the conventional methods. In order to tap into the wealth of agronomically important genes present in the alien species without any yield penalty, an efficient method of directed gene transfers is therefore needed.

Generally, strict diploid-like chromosome pairing is observed in fertile polyploids even though homoeologues may share a high level of sequence similarity due to conserved gene order. Homology search is one of the most poorly understood mechanisms in the meiotic process. Homology search during meiosis is more challenging in “younger” polyploids such as allohexaploid wheat, where differences between homologues and homoeologues are less pronounced, entailing an efficient homology search mechanism to ensure diploid-like pairing behavior. The Ph1 gene of wheat ensures strict diploid-like pairing even though the homoeologues are similar in gene content and order, and thus are capable of pairing.

Since its discovery in 1958, various studies have implicated the Ph1 gene in many different meiotic processes. While studying somatic association of chromosomes in pre-meiotic cells, the Ph1 gene was suggested to be involved in ensuring strict homologous pairing by suppressing pre-meiotic homoeologous chromosome association. Analyses of the published data implicate the 5D copy of the Ph1 gene in initial chromosome pairing of both homologues as well as homoeologues, as asynapsis was observed in the absence of the 5D copy but not the 5A or the 5B copies. Absence of chromosome 5A had essentially no effect on homoeologous chromosome pairing (HECP). While the 5B copy of the Ph1 gene was shown to specifically regulate diploid-like pairing, various lines lacking chromosome 5B, its long-arm, or the segment carrying Ph1 locus all showed increased HECP. Function of the 5B copy in differentiating homologues from homoeologues was further supported by the fact that four copies of neither 5A nor 5D were able to restore normal chromosome pairing in the absence of chromosome 5B. Multivalents and other types of higher order pairing observed in the absence of the 5B copy, were not observed in the absence of the 5A copy and were not as robust in the absence of the 5D copy. Along with asynapsis, lack of 5D exhibited frequent bivalent interlocking, and rare multivalents. A minimum four copies of the 5A copy were needed to compensate for the absence of the 5D copy, suggesting that the two copies share a common function with the 5A copy having a weaker effect.

Lack of the Ph1 gene results in multivalents during metaphase I (MI) of meiosis resulting in partial sterility. Conversely, six doses of the gene in tri-isosomic line of 5BL resulted in interlocking of the bivalents and reduced chiasmata frequency even among homologues along with rare multivalents. Several other genes promoting or suppressing HECP have also been reported although their effect is difficult to measure in the presence of the Ph1 gene. Ph1-like genes were also reported in other sexually propagating polyploids including Avena sativa, Festuca arundinacea, Brassica napus, Gossypium hirsutum, G. barbadense, and in some diploids including Lolium perenne, L. multiflorum and L. rigidum.

Therefore, there exists a need for new compositions and methods for providing efficient methods of transferring and manipulating genes in plants without undesirable carry-over genes. Accordingly, the present disclosure provides improved compositions and methods for reducing gene expression of a Ph1 gene in a plant and identifies gene(s) responsible for the Ph1 gene-like function.

The compositions and methods according to the present disclosure provide several advantages compared to other compositions and methods known in the art. First, the compositions and methods described herein provide silencing of a Ph1 gene (e.g., “C-Ph1” to represent “candidate for the Ph1 gene of wheat”) to result in advantageous pairing between homoeologous chromosomes. Second, the compositions and methods described herein provide for the induction of pairing and recombination between any related chromosomes to transfer value added genes from related species and genera into crop plants. Third, the compositions and methods described herein can be accomplished either by stable RNAi, transient RNAi, or using any other methods of gene silencing. Finally, since Ph1 gene function is also conserved in other plant species, the compositions and methods described herein can be equally applied to other plant species including, but not limited to, wheat, maize, rice, brassica, cotton, barley, and Brachypodium.

The following numbered embodiments are contemplated and are non-limiting:

1. A plant or plant part comprising a homoeologous recombination of chromosomes, wherein the plant or plant part comprises reduced expression of a Ph1 gene.

2. The plant or plant part of clause 1, wherein the plant or plant part is a hybrid plant or plant part.

3. The plant or plant part of clause 1, wherein the plant or plant part is a transgenic plant or plant part.

4. The plant or plant part of any of clauses 1 to 3, wherein the plant or plant part is wheat.

5. The plant or plant part of any of clauses 1 to 3, wherein the plant or plant part is a wheat relative.

6. The plant or plant part of any of clauses 1 to 3, wherein the plant or plant part is maize.

7. The plant or plant part of any of clauses 1 to 3, wherein the plant or plant part is rice.

8. The plant or plant part of any of clauses 1 to 3, wherein the plant or plant part is barley.

9. The plant or plant part of any of clauses 1 to 3, wherein the plant or plant part is Brachypodium.

10. The plant or plant part of any of clauses 1 to 3, wherein the plant or plant part is a post transcriptional Ph1 gene silenced plant or plant part.

11. The plant or plant part of any of clauses 1 to 10, wherein the reduced expression of a Ph1 gene is between 22% and 83% reduction in gene expression compared to a negative control.

12. The plant or plant part of clause 11, wherein the reduction in gene expression is via transcriptional suppression.

13. The plant or plant part of clause 11, wherein the reduction in gene expression induces homoeologous chromosome pairing.

14. The plant or plant part of any of clauses 1 to 13, wherein the Ph1 gene is C-Ph1.

15. The plant or plant part of clause 1, wherein the plant is wheat and wherein the Ph1 gene is the 5A gene copy.

16. The plant or plant part of clause 1, wherein the plant is wheat and wherein the Ph1 gene is the 5B gene copy.

17. The plant or plant part of clause 1, wherein the plant is wheat and wherein the Ph1 gene is the 5D gene copy.

18. The plant or plant part of any of clauses 1 to 17, wherein the plant or plant part is inoculated with a vector construct comprising a nucleotide sequence selected from the group consisting of SEQ ID. NO:4, SEQ ID. NO:5, SEQ ID. NO:6, and SEQ ID. NO:7.

19. A method of reducing gene expression of a Ph1 gene in a plant, said method comprising the step of silencing the Ph1 gene in a chromosome of the plant.

20. The method of clause 19, wherein the silencing is virus induced gene silencing (VIGS).

21. The method of clause 19, wherein the silencing is transient silencing.

22. The method of clause 21, wherein the transient silencing is RNAi silencing.

23. The method of clause 19, wherein the silencing is stable silencing.

24. The method of clause 23, wherein the stable silencing is RNAi silencing.

25. The method of any one of clauses 19 to 24, wherein the plant is a polyploid plant.

26. The method of clause 25, wherein the polyploid plant is polyploid wheat.

27. The method of any one of clauses 19 to 24, the plant is wheat.

28. The method of any one of clauses 19 to 24, the plant is a wheat relative.

29. The method of any one of clauses 19 to 24, the plant is maize.

30. The method of any one of clauses 19 to 24, the plant is rice.

31. The method of any one of clauses 19 to 24, the plant is barley.

32. The method of any one of clauses 19 to 24, the plant is Brachypodium.

33. The method of any one of clauses 19 to 24, the Ph1 gene is the 5A gene copy.

34. The method of any one of clauses 19 to 24, the Ph1 gene is the 5B gene copy.

35. The method of any one of clauses 19 to 24, the Ph1 gene is the 5D gene copy.

36. The method of any one of clauses 19 to 24, the method induces pairing between a wheat chromosome and a wheat-related chromosome.

37. The method of any one of clauses 19 to 24, the method induces recombination of a wheat chromosome and a wheat-related chromosome.

38. The method of any one of clauses 19 to 24, the method transfers one or more genes into the plant.

39. The method of clause 38, wherein the transfer is from the wheat-related chromosome.

40. The method of clause 38, wherein the transfer is via homoeologous chromosome pairing.

41. The method of any one of clauses 19 to 40, wherein the method results in formation of a transgenic plant.

42. A vector construct comprising a nucleotide sequence selected from the group consisting of SEQ ID. NO:4, SEQ ID. NO:5, SEQ ID. NO:6, and SEQ ID. NO:7.

43. The vector construct of clause 42, wherein the vector construct is a hairpin construct.

44. The vector construct of clause 42, wherein the vector construct is an antisense construct.

45. The vector construct of any of clauses 42-44, wherein the nucleotide sequence selected from the group consisting of SEQ ID. NO:5, SEQ ID. NO:6, and SEQ ID. NO:7.

46. The vector construct of clause 43, wherein the vector construct is pγ.C-Ph1hp1.

47. The vector construct of clause 43, wherein the vector construct is pγ.C-Ph1hp2.

48. The vector construct of clause 44, wherein the vector construct is pγ.C-Ph1as.

49. The vector construct of clause 43, wherein the vector construct is pHellsgate8 1-1

50. The vector construct of any of clauses 42-49, wherein the nucleotide sequence comprises a nucleotide sequence selected from the group consisting of SEQ ID. NO:6, and SEQ ID. NO:7.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a wheat-rice comparison demonstrating alignment of the Ph1 gene region to rice chromosome 9 and BAC scaffolds of wheat 5B and 5D chromosomes. Genomic sequence of rice chromosome 9, from coordinate 18,162,398 bp to 18,615,877 bp corresponding to ‘Ph1 gene region’ is shown in light pink. The rice chromosome region between bcd1088 and cdo1090c is drawn to scale. The genes present in these coordinates were mapped to wheat BAC scaffolds (retrieved from NCBI database) using BLAST program. Various BACs are drawn in light blue and pink for chromosomes 5D and 5B, respectively. Partial overlap is drawn as pink for 5B and light blue for 5D. Genes and DNA markers assigned to wheat BAC scaffolds and rice chromosome 9 are shown in green and red, respectively. Within a BAC, order and position of these genes may vary. Dark blue on rice chromosome 9 represent the candidate genes involved in meiosis. Individual BAC is represented by thin black bar oriented towards one direction. Red star represents the C-Ph1 gene marked on rice chromosome 9, 5D and the 5B wheat contigs (IWGSC_CSS_5BL_scaff_10882589 retrieved from IWGSC sequence database is shown in magenta). Extreme left bar represents long arm of chromosome 5B demarcating the deletion break points. The C-bands on this bar are shown in orange and Ph1 region in dark green.

FIG. 2 shows a cytogenetic analysis demonstrating different levels of C-Ph1 gene silencing in RNAi plants compared with ph1b. Chromosome spreads of PMCs from ph1b, BW (negative control), and four RNAi plants showing different levels of gene silencing. “Exp” denotes the normalized transcript expression levels (%) relative to BW, using the delta-delta threshold cycle (Ct) method, observed by quantitative real-time PCR analysis. Aberrant pairing (%) denotes the percentage of cells exhibiting aberrant chromosome pairing. “Multivalents/cell” denotes the average number of multivalents per cell, and the range is given in parentheses. Chromosome (Chr.) clustering and misalignment phenotype are represented by “+” and “−,” where (+) indicates increased severity levels and (−) indicates decreased severity levels. (Scale bar, 5 μm.)

FIG. 3 shows a comparison of chromosomal pairing in the VIGS and RNAi silenced plant with ph1b and CS. Chromosome spreads of meiotic MI pollen mother cells (PMCs) from the VIGS, RNAi silenced plant, ph1b and CS with no inoculation.

FIG. 4 shows multivalent formation in the Arabidopsis silenced plants. Each image is a flat projection across the entire nucleus. Chromosomes were counterstained with DAPI (red); centromeric probe was labeled with cyanine-5 (green). Normal meiosis progression from leptotene to late pachytene leading to formation of five bivalents in the wild-type (A-C). Centromere coupling during leptotene (D), multivalent formation in zygotene (E) (inset showing quadrivalent formation) in the silenced plants. Centromere coupling in pachytene leading to formation of two clusters of centromeres (F) instead of five pairs in wild type (C). (Scale bar, 5 μm)

FIG. 5 shows the chromosomal pairing (CP) and C-Ph1 gene expression analysis in the VIGS and RNAi-silenced plants. (A) Chromosome spreads of PMCs at MI from FES and MCS controls and C-Ph1 silenced plants VIGS-5 and VIGS-7. CP (chromosome pairing) analysis shows (i) the percentage of cells exhibiting aberrant pairing and the total number of cells analyzed (given in parenthesis) and (ii) the average number of multivalents per cell, with the range given in parentheses. EXP (expression) denotes the transcript expression levels (%) in the C-Ph1 silenced plants relative to the FES control, as observed by quantitative real-time PCR analysis. (B) Chromosome spreads of PMCs of control (BW) and C-Ph1 silenced RNAi plants indicating CP and EXP. (Scale bar, 5 μm.)

FIG. 6 shows a gene specific qRT-PCR analysis in the VIGS silenced plants, MCS and FES. The Y-axis denotes the transcript gene expression levels normalized to Actin using the delta-delta Ct method in the spike tissue (4-5 cm) analyzed, observed from quantitative real-time PCR.

FIG. 7 shows a gene specific qRT-PCR analysis in the RNAi and control plants. The Y-axis denotes the transcript gene expression levels normalized to Actin using the delta-delta Ct method in the spike tissue (4-5 cm) analyzed, observed from quantitative real-time PCR.

FIG. 8 shows structural differences among the C-Ph1 gene homoeologues in hexaploid wheat. Nucleotide sequences of cloned CS C-Ph1-5B, its splice variant (C-Ph1-5B^{alt}), and C-Ph1-5D and C-Ph1-5A copies were aligned to each other; the differences are drawn to scale (1=1 nucleotide). The symbols ▴ and ▾ represent deletions and insertions in the sequences, respectively. Insertions and deletions were determined by majority consensus rule. The shaded region in C-Ph1-5B and C-Ph1-5B^{alt} represents a corresponding region similar to the C-Ph1-5D and C-Ph1-5A sequences; the nucleotide sequence is not translated as a protein (predicted) but forms a part of the UTR. The colored bars below C-Ph1-5B represent VIGS and RNAi oligos, denoted by as (antisense), hp1 (hairpin 1), hp2 (hairpin 2), and RNAi, respectively. The gray dots at the end of C-Ph1-5A exon II represent deletion/insertion not present in the C-Ph1-5B and C-Ph1-5D sequences.

FIG. 9 shows 3D models for protein structure and functions. The protein structures were predicted using I-TASSER online platform and matched with BioLiP protein function database. PDBeFold was used to compare the 3D protein structures and percent similarities between protein structures were predicted. 5A, 5B and 5D refer to the protein structures of the identified gene homoeologues while 5B^{alt} refers to the protein structure of the spliced variant of 5B copy.

FIGS. 10A-10E show C-Ph1 gene expression pattern in various tissues and substaged meiotic anthers. (FIG. 10A) Chromosome spreads of PMCs from CS denoting various stages of meiosis. (Scale bar, 5 μm.). (FIG. 10B) Quantitative expression analysis using gene-specific primers in the root (R), leaf (L), flag leaf (FL), 3- to 5-cm spike (I to MI), and 6- to 8-cm spike (MII to T), as well as at anthesis (AN) and 5 days post-anthesis (SDPA). (FIG. 10C) Quantitative expression analysis at interphase (I), prophase I (P), late prophase I and metaphase I (M), anaphase I (A), dyad (D), and tetrad (T). The Y-axis in (B) and (C) denotes the normalized mRNA levels using the delta-delta Ct method. (FIG. 10D) Tissue- and stage-specific expression of homoeologues analyzed by single-strand conformation polymorphism (SSCP) analysis. (FIG. 10E) Meiotic stage-specific expression of homoeologues in the different sub-stages of meiosis, as mentioned in (C).

FIG. 11 shows gene specific qRT-PCR analysis in the wheat homoeologous group 5 NT lines, Ph1 mutants and 5B-specific deletion lines. The Y-axis denotes the transcript gene expression levels normalized to Actin using the delta-delta Ct method in the spike tissue (4-5 cm) of the lines analyzed, observed from quantitative real-time PCR. The 5B-specific primer (material and methods) was used for the analysis.

FIG. 12 shows gene mapping of Cdc2-4 using wheat homoeologous group 5 NT lines, Ph1 mutants and 5B-specific deletion lines. The gene is amplified using sequence tagged site (STS) primers for wheat Cdc2-4 gene. The PCR product is resolved on Roche LightCycler® 480 (Roche Diagnostics, USA) using Melt Curve analysis and 2% agarose gel.

FIG. 13 shows chromosome spreads of meiotic metaphase I pollen mother cells of VIGS treated Cdc2-4, MCS, FES and CS control plants. MCS is the positive control, virus construct carrying 121-bp antisense fragment of the multiple cloning site (MCS) from pBluescript K/S (Stratagene). FES is the negative control, plants rubbed with the abrasive agent only.

Various embodiments of the invention are described herein as follows. In one embodiment described herein, a plant or plant part comprising a homoeologous recombination of chromosomes is provided. The plant or plant part is specified wherein it comprises reduced expression of a Ph1 gene. In another embodiment described herein, a method of reducing gene expression of a Ph1 gene in a plant is provided. The method comprises the step of silencing the Ph1 gene in a chromosome of the plant. In yet another embodiment described herein, a vector construct is provided. The vector construct comprises a nucleotide sequence selected from the group consisting of SEQ ID. NO:4, SEQ ID. NO:5, SEQ ID. NO:6, and SEQ ID. NO:7.

In one embodiment of the present disclosure, a plant or plant part comprising a homoeologous recombination of chromosomes is provided, wherein the plant or plant part reduced expression of a Ph1 gene. As used herein, the term “plant” includes a whole plant and any descendant, cell, tissue, or part of a plant. The term “plant parts” include any part(s) of a plant, including, for example and without limitation: seed (including mature seed and immature seed); a plant cutting; a plant cell; a plant cell culture; a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. In contrast, some plant cells are not capable of being regenerated to produce plants. Regenerable cells in a plant cell or tissue culture may be embryos, protoplasts, meristematic cells, callus, pollen, leaves, anthers, roots, root tips, silk, flowers, kernels, ears, cobs, husks, or stalks.

As used herein, the term “homoeologous” refers to related chromosomes (e.g., chromosomes from wheat and from wild relatives) and is different from “homologous” chromosomes, which represent pair of chromosomes that normally pair in a normal plant (e.g., present in normal wheat). The recombination of chromosomes with respect to plants is well known to a person of ordinary skill in the art. “Recombination” refers to the reassortment of sections of DNA or RNA sequences between two DNA or RNA molecules.

As used herein, the term “reduced expression” refers to a numerical reduction in expression of the gene, for example compared to a negative control or to a wild type. In some embodiments, reduced expression can be identified using PCR analysis, for example quantitative real-time PCR analysis. In some embodiments, a reduction in expression can range from about 5% to about 50%. In other embodiments, a reduction in expression can range from about 7% to about 22%. In some embodiments, a reduction in expression can range from about 20% to about 44%. In other embodiments, a reduction in expression can range from about 50 to about 85%. In one embodiment, the reduced expression of a Ph1 gene is between 22% and 83% reduction in gene expression compared to a negative control. In one embodiment, the reduced expression of a Ph1 gene is between 22% and 44% reduction in gene expression compared to a negative control.

As used herein, a “Ph-1” gene refers to the Pairing homoeologous 1 (Ph1) gene, which ensures strict diploid-like pairing even though the homoeologues of varying plants are similar in gene content and order, and thus are capable of pairing.

In some embodiments, the plant or plant part is a hybrid plant or plant part. As used herein, the term “hybrid” refers to any individual cell, tissue, plant, or plant part resulting from a cross between parents that differ in one or more genes. In some embodiments, the plant or plant part is a transgenic plant or plant part. As used herein, the term “transgenic” refers to cells, cell cultures, organisms, plants, and progeny of plants which have received a foreign or modified gene by one of the various methods of transformation, wherein the foreign or modified gene is from the same or different species than the species of the plant, or organism, receiving the foreign or modified gene.

In some embodiments, the plant or plant part is wheat. In other embodiments, the plant or plant part is a wheat relative. As used herein, the term “wheat relative” is well known in the art as a species that is genetically similar to wheat, for example rye. In yet other embodiments, the plant or plant part is maize. In some embodiments, the plant or plant part is rice. In other embodiments, the plant or plant part is barley. In yet other embodiments, the plant or plant part is Brachypodium.

In one embodiment, the plant or plant part is a post transcriptional Ph1 gene silenced plant or plant part. The post transcriptional Ph1 gene silenced plant or plant part is demonstrated in the examples of the present disclosure. In some embodiments, the post transcriptional Ph1 gene silenced plant or plant part is a RNAi-5 plant or plant part.

In some embodiments, the reduction in gene expression is via transcriptional suppression. In other embodiments, the reduction in gene expression induces homoeologous chromosome pairing.

In various embodiments, the Ph1 gene is C-Ph1. The present disclosure provides identification of the C-Ph1 gene expressing exclusively during MI, and whose silencing resulted in formation of multivalents similar to the Ph1 gene mutations.

The genomic sequence of C-Ph1 is presented as follows as SEQ ID NO:1:

(SEQ ID NO: 1)
5′ATGGCGCGCCTCCTCGTTCTCGCCGTCACGGCCACGGTTCTCATGGTG
CGAAAGAGCTAGCTAGTAGAGCCGGCCATGCATGGCACAGATACCTATCA
TACACGACTGAATTTTGTCTTGTCAAACATTTCATCGGTGCGTTTTTTTT
GTCTCTCCAGGCTCAATCCGGGCAGCCGGCGTCTGCTGCGCGCCGCCCTG
CCCGACGCCCCCATGCCGGACGCCATCCTCGAGCTCCTGCCCCANTTTGA
TCACCACGCATCAACGGAACAGGGTACTGATTTTGTGGCCATCTTCCGAT
GGAAGGCCGTCTCTCTCTAGCTCACCCACGTGCCTTGCTGCATGAATGCA
GAGAAAGACACTCCGGAAGGCGCGGTCGAGGACGTGGAGGACAAGGACCC
GCCGCCGCCCATGAACTTCAACTACGACTACGATGACGCCTTGCCCCGGA
GCGAAACCACCAGCGCCCCCTCCCCCGACGTCCTACTGAACCGCGCCGCC
GTCGTCCGCAACGTCGCCACGCCGTCGTCGGCGGTGTTCTTCCTCGAGGA
CGCGGTGCGCGTCCGGGAGAGCCTGCCCTTCCACAGGATCCATCGGGCCA
CCGGCGCTGCCGAGGCGTCGGCAGAACAGCCGCTGGAGCTGTACACTGTG
CACTCCGTGAGGGCGGTCGAGGGGTCCAATTTCATCCTGTGCCGGGGTGA
AGCCGGCGAAGGGGCCGTGTACGGGTGCCGCGCAACCGGCCCGGCGAGGG
CCTACGTCCTGGCCCTGGCCGGCGAGCGCGGGGACGTGACGATGACCGCG
GTTGCCGTGTGCCGCACCGACGCATCCCGATGGGACCCGGAGCACGCCGC
CTTCCGGCTCCTGGGCGTGAAGCCCGGCGGCGCGGCGGTCTGCCACGCGG
TGCGGGACGCGCAGCTCCTGCCGGCCATGAACGGGAAGAGCCCCGTCGCC
AACTAA3′.

Although the C-Ph1 gene has three homoeologous copies, the 5B copy has diverged in sequence from the other two copies. Heterologous gene silencing of the Arabidopsis homologue of the C-Ph1 gene also confirmed the role of the C-Ph1 gene in chromosome pairing. Molecular characterization of the C-Ph1 gene provides for the development of new tools and strategies for breeding activities by allowing precise alien introgressions.

The sequence of C-Ph1 CDS is presented as follows as SEQ ID NO:2:

(SEQ ID NO: 2)
5′ATGAATGCAGAGAAAGACACTCCGGAAGGCGCGGTCGAGGACGTGGAG
GACAAGGACCCGCCGCCGCCCATGAACTTCAACTACGACTACGATGACGC
CTTGCCCCGGAGCGAAACCACCAGCGCCCCCTCCCCCGACGTCCTACTGA
ACCGCGCCGCCGTCGTCCGCAACGTCGCCACGCCGTCGTCGGCGGTGTTC
TTCCTCGAGGACGCGGTGCGCGTCCGGGAGAGCCTGCCCTTCCACAGGAT
CCATCGGGCCACCGGCGCTGCCGAGGCGTCGGCAGAACAGCCGCTGGAGC
TGTACACTGTGCACTCCGTGAGGGCGGTCGAGGGGTCCAATTTCATCCTG
TGCCGGGGTGAAGCCGGCGAAGGGGCCGTGTACGGGTGCCGCGCAACCGG
CCCGGCGAGGGCCTACGTCCTGGCCCTGGCCGGCGAGCGCGGGGACGTGA
CGATGACCGCGGTTGCCGTGTGCCGCACCGACGCATCCCGATGGGACCCG
GAGCACGCCGCCTTCCGGCTCCTGGGCGTGAAGCCCGGCGGCGCGGCGGT
CTGCCACGCGGTGCGGGACGCGCAGCTCCTGCCGGCCATGAACGGGAAGA
GCCCCGTCGCCAACTAA3′.

The sequence of C-Ph1-Alt CDS is presented as follows as SEQ ID NO:3:

(SEQ ID NO: 3)
5′ATGCCGGACGCCATCCTCGAGCTCCTGCCCCAGTTTGATCACCACGCA
TCAACGGAACAGGAGAAAGACACTCCGGAAGGCGCGGTCGAGGACGTGGA
GGACAAGGACCCGCCGCCGCCCATGAACTTCAACTACGACTACGATGACG
CCTTGCCCCGGAGCGAAACCACCAGCGCCCCCTCCCCCGACGTCCTACTG
AACCGCGCCGCCGTCGTCCGCAACGTCGCCACGCCGTCGTCGGCGGTGTT
CTTCCTCGAGGACGCGGTGCGCGTCCGGGAGAGCCTGCCCTTCCACAGGA
TCCATCGGGCCACCGGCGCTGCCGAGGCGTCGGCAGAACAGCCGCTGGAG
CTGTACACTGTGCACTCCGTGAGGGCGGTCGAGGGGTCCAATTTCATCCT
GTGCCGGGGTGAAGCCGGCGAAGGGGCCGTGTACGGGTGCCGCGCAACCG
GCCCGGCGAGGGCCTACGTCCTGGCCCTGGCCGGCGAGCGCGGGGACGTG
ACGATGACCGCGGTTGCCGTGTGCCGCACCGACGCATCCCGATGGGACCC
GGAGCACGCCGCCTTCCGGCTCCTGGGCGTGAAGCCCGGCGGCGCGGCGG
TCTGCCACGCGGTGCGGGACGCGCAGCTCCTGCCGGCCATGAACGGGAAG
AGCCCCGTCGCCAACTAA3′.

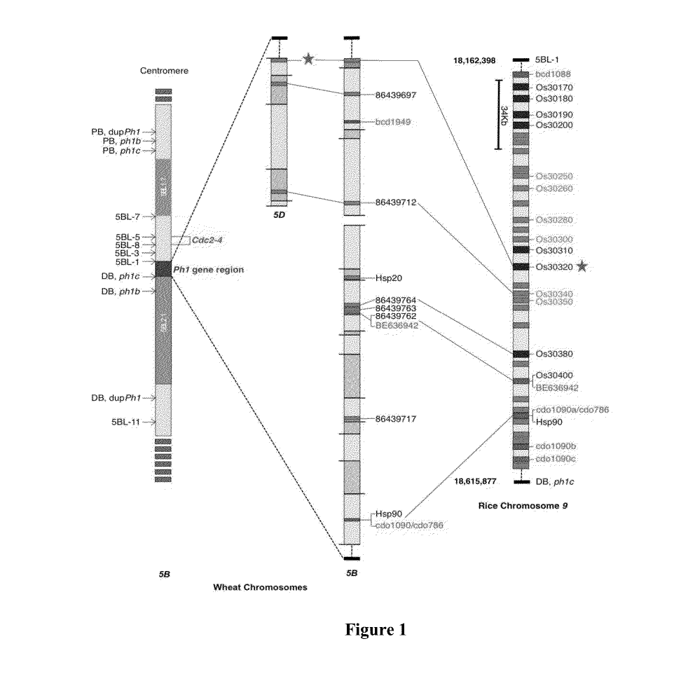

Ph1 gene mutants in tetraploid (ph1c) and in hexaploid (ph1b) wheat were shown to be interstitial deletions respectively involving □0.84 μm and □1.05 μm region around the gene (see FIG. 1). Physical mapping localized the gene to a □2.5 Mb chromosomal region referred to as “Ph1 gene region,” bracketed by distal breakpoint of ph1c (DB, ph1c) deletion on the distal end and breakpoint of deletion line 5BL-1 on the proximal end (see FIG. 1). Various marker enrichment efforts identified nine markers for the region. Detailed micro-synteny analyses and comparative mapping identified a 450 kb region of rice chromosome 9. The corresponding rice region contained 91 genes.

In some embodiments, the plant is wheat and the Ph1 gene is the 5A gene copy. In other embodiments, the plant is wheat and the Ph1 gene is the 5B gene copy. In yet other embodiments, the plant is wheat and the Ph1 gene is the 5D gene copy.

In various embodiments, the plant or plant part is inoculated with a vector construct comprising a nucleotide sequence. In some embodiments, the nucleotide sequence comprises a C-Ph1-RNAi sequence (SEQ ID NO:4) as follows:

(SEQ ID NO: 4)
5′CGTCCTACTAAACCGCGCTGCCGTCGTCACGCCGTCGTCGACGGTGTT
CTTCCTCGAGGACGCGGTGCGCGTCGGGGAGAGCCTGCCCTTCCACAGGA
TCCATCGGGCCACCGCCGCCGCCGAGGCGTCGGCAGAGCAGCCGCTGGAG
CTGTACACCGTCCGCTCCGTGAGGGCGGTCGAGGGGTCCAGTTTCGTCCT
GT3′.

In other embodiments, the nucleotide sequence comprises a C-Ph1-VIGS-as sequence (SEQ ID NO:5) as follows:

(SEQ ID NO: 5)
5′CGGTCGAGGCCGTGGAGGACAAGGACCCGCCGCCGCCCATGAACTTCA
ACTACGACTACGACGACGCCTTGCCCCGGAGCGAAGCCACCAGCGCCCC
C3′.

In yet other embodiments, the nucleotide sequence comprises a C-Ph1-VIGS-hp1 sequence (SEQ ID NO:6) as follows:

(SEQ ID NO: 6)
5′CGCTGCCGTCGTCACGCCGTCGTCGACGGTGTTCTTCCTCGAGGA3′.

In yet other embodiments, the nucleotide sequence comprises a C-Ph1-VIGS-hp2 sequence (SEQ ID NO:7) as follows:

(SEQ ID NO: 7)
5′CGTGAGGGCGGTCGAGGGGTCCAGTTTCGTCCTGTGCCGG3′.

In another embodiment of the present disclosure, a method of reducing gene expression of a Ph1 gene in a plant is provided. The method comprises the step of silencing the Ph1 gene in a chromosome of the plant. The previously described embodiments of the plant or plant part are applicable to the method of reducing gene expression of a Ph1 gene described herein. As used herein, the term “silencing” refers to a reduction of expression of a gene in a plant. Gene silencing can occur during either transcription or translation. In some embodiments, the silencing is virus induced gene silencing (VIGS).

In various aspects, the silencing is transient silencing. In some embodiments, the transient silencing is RNA interference (RNAi) silencing. In other aspects, the silencing is stable silencing. In some embodiments, the stable silencing is RNAi silencing. RNAi using stable transgenic wheat plants (Fu et al., Transgenic Res., 2007; 16(6):689-701; herein incorporated by reference in its entirety) and transient silencing using Virus Induced Gene Silencing (VIGS) have been used to silence resistance genes in wheat (Scofield et al., Plant Physiology, 2005; 138(4): 2165-2173; herein incorporated by reference in its entirety).

In some embodiments, the plant is a polyploid plant. As used herein, the term “polyploid plant” refers to a plant containing more than two paired (homologous) sets of chromosomes. In various embodiments, the polyploid plant is polyploid wheat.

In various aspects, the method induces pairing between a wheat chromosome and a wheat-related chromosome. In certain aspects, the method induces recombination of a wheat chromosome and a wheat-related chromosome. In certain embodiments, the method transfers one or more genes into the plant. In some embodiments, the transfer is from the wheat-related chromosome. In other embodiments, the transfer is via homoeologous chromosome pairing. In certain aspects, the method results in formation of a transgenic plant.

In yet another embodiment of the present disclosure, a vector construct is provided. The vector construct comprises a nucleotide sequence comprises a sequence selected from the group consisting of SEQ ID. NO:4, SEQ ID. NO:5, SEQ ID. NO:6, and SEQ ID. NO:7. In various aspects, the nucleotide consists of a sequence selected from the group consisting of SEQ ID. NO:4, SEQ ID. NO:5, SEQ ID. NO:6, and SEQ ID. NO:7. In some embodiments, the nucleotide sequence comprises SEQ ID. NO:4. In other embodiments, the nucleotide sequence comprises SEQ ID. NO:5. In yet other embodiments, the nucleotide sequence comprises SEQ ID. NO:6. In some embodiments, the nucleotide sequence comprises SEQ ID. NO:7. In some embodiments, the nucleotide sequence consists of SEQ ID. NO:4. In other embodiments, the nucleotide sequence consists of SEQ ID. NO:5. In yet other embodiments, the nucleotide sequence consists of SEQ ID. NO:6. In some embodiments, the nucleotide sequence consists of SEQ ID. NO:7.

While the invention is susceptible to various modifications and alternative forms, specific embodiments are herein described in detail. It should be understood, however, that there is no intent to limit the invention to the particular forms described, but on the contrary, the intention is to cover all modifications, equivalents, and alternatives falling within the scope of the invention.

### EXAMPLE 1

### Identification of C-pH1 (Candidate pH1)

During MI, short microtubules radiate out from the microtubule organizing centers within an hour of nuclear membrane breakdown and form a barrel shaped bipolar spindle with the kinetochore assembly. The centromere-microtubule interaction for meiosis is critical for proper alignment of paired chromosomes on the MI plate. Previously, the 5B copy of the Ph1 gene was suggested to ensure strict homologous pairing by regulating proper microtubule-centromere interaction and dynamics. Measured as sensitivity of spindle to anti-microtubule drugs, the Ph1 gene affected the dynamics of spindle assembly thereby ensuring proper arrangement of chromosomes along the MI plate. The spindle assembly was observed to be highly unstable in the absence of the 5B copy and its stability increased proportionately with the increase in the 5B copy number. The Ph1 gene was reported to regulate microtubule-centromere interaction by modulating phosphorylation of tubulin proteins primarily during late prophase I to MI. Involvement of centromeres in the Ph1 gene function was further supported by the observation that in comparison to normal CS, transverse division of univalents was significantly different in the absence of the 5B copy of the Ph1 gene.

In addition, it is possible that the role of C-Ph1-5B gene in microtubule-centromere interaction could be one of its overlapping functions with other two homeologous copies of the gene. Accordingly, the unique function of Ph1 gene in early pachytene cannot be disregarded at this point in time.

The following criteria were used to select the potential Ph1 gene candidates from the 91 genes present in the 450 kb rice region: 1) Gene expressing during meiosis, and 2) Genes involved in chromatin reorganization, microtubule attachment, acetyltransferases, methyltransferases, and DNA binding. This detailed bioinformatic analyses identified 26 genes for further characterization. Virus induced gene silencing (VIGS) was optimized for wheat meiosis using the disrupted meiotic cDNA1 (DMC1), lack of which results in mostly univalents at MI. VIGS of TaDMC1 with an antisense construct resulted in an average of 37.2 univalents and 2.4 bivalents.

All plants were propagated in 4 or 6-inch pots using Sunshine#1 potting mixture (SunGro Horticulture, Bellevue, Wash., USA) supplemented with 14 g Nutricote 14-14-14 fertilizer (Plantco Inc., Brampton, Ontario, Canada). Plants were grown under 16 hr light at 500-700 μmol m-2s-1 in a Conviron PGR15 growth chamber equipped with high-intensity discharge lamps.

Plant material used in this example include wild-type hexaploid wheat (T. aestivum cv. Chinese Spring and cv. Bob White); a Chinese Spring mutant lacking the Ph1 locus (ph1b); wheat homoeologous group 5 NT lines, and a series of 5BL deletion lines. Based on the efficient utilization of the cv. CS for various genetic and molecular studies including VIGS, it was selected as an ideal cultivar for VIGS. CS, NT and deletion lines were used for mapping and cloning experiments. Bobwhite was used for RNAi experiments as it can be efficiently transformed using the Agrobacterium-mediated gene transfer.

Except for about 4-6% of the MI cells that usually show aberrant chromosome pairing including multivalents, 21 bivalents were observed in the wild-type wheat cultivars Chinese Spring (CS) and Bobwhite (BW). These data are shown in Table A and Table B below.

TABLE A
Chromosome pairing analysis in CS and ph1 mutant and deletion lines.

| | | Average | Average | Average | % cells |
|---|---|---|---|---|---|
| | Number of | number of | number of | number of | showing |
| | cells | univalent/ | bivalents/ | multivalents/ | aberrant |
| Plant-type | analyzed | cell | cell | cells | pairing |
| CS | 50 | 0.18 | 20.12 | 0.04 | 4 |
| ph1b | 50 | 1.93 | 16.9 | 1.29 | 60 |

TABLE B
Chromosome pairing at metaphase I of the transgenics and bobwhite
(control) plants. Aberrant chromosome pairing indicates the percentage
of cells with multivalents, misalignment and chromosome clumping.
Bivalents are indicated as average values of rod and ring chromosomes.
Cell number indicates the total number of cells analyzed. The gene
expression refers to the transcript expression levels (%) relative to
the control (Bobwhite), observed from quantitative real-time PCR.
The transcript is normalized to Actin using the delta-delta Ct method.

| | Aberrant | | | | |
|---|---|---|---|---|---|
| | chromosome | Bivalents | | | Gene |
| | pairing | (Average) | | | expression |
| Plant | (%) | Rod | Ring | Cell number | (%) |
| RNAi-5 | 22 | 2.4 | 9.68 | 27 | 56 |
| RNAi-3 | 14.28 | 1.62 | 10.07 | 35 | 78.39 |
| RNAi-4 | 90 | 1.8 | 14.27 | 40 | 17.42 |
| RNAi-6 | 71.73 | 1.75 | 11.38 | 46 | 49.06 |
| RNAi-2 | 86.66 | 1.65 | 10.57 | 30 | 22.38 |
| RNAi-1 | 76 | 1.63 | 11.7 | 25 | 30.2 |
| RNAi-7 | 6.25 | 3.87 | 6.8 | 16 | 92.37 |
| Control (BW) | 6.0 | 1.68 | 19.72 | 50 | 100 |

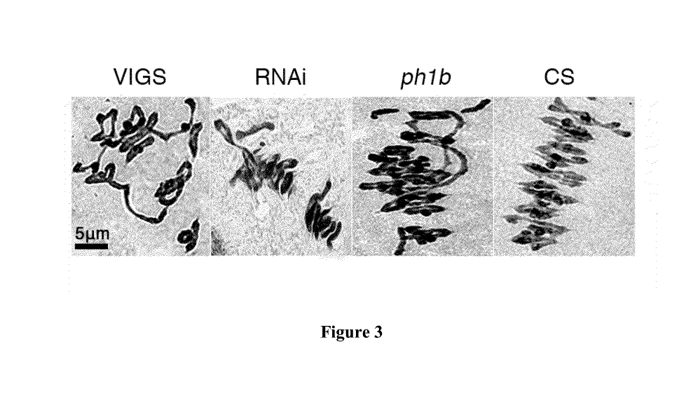

In the ph1b mutant, about 60% of the cells showed the aberrant chromosome pairing with an average of 1.29 multivalents and 1.93 univalents per cell (see Table A). The higher number of univalents observed in the ph1b mutant may be due to the combined effect of other genes present in the □1.05 μm chromosomal region deleted in the mutant line. At least two genes (LOC_Os09g30310 and LOC_Os09g31310) have been identified in the deleted part of ph1b, silencing of which resulted in 2-8 univalents. About 8% of the ph1b cells showed bivalent interlocking. The ph1b (see FIG. 3) and other Ph1 mutant and deletion lines show relatively normal chromosome alignment on the MI plate and chromosome clumping is usually not observed.

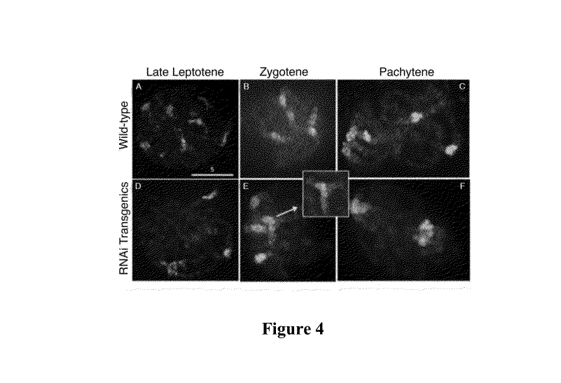

A hairpin construct for the C-Ph1 gene, pγ.Ph1hp1 (91-bp) was designed from wheat EST BE498862. In addition, the antisense construct pγ.Ph1as (98-bp), and hairpin construct pγ.Ph1hp2 (110-bp) were also designed from the conserved regions of the full-length gene sequence (see FIG. 4). The antisense construct of Cdc2-4 gene was 96-bp long targeting 34176-34271 bp of the Cdc2-4B gene (start: 34093 bp-end: 35118 bp on AM050673) and was designed following the criteria described above.

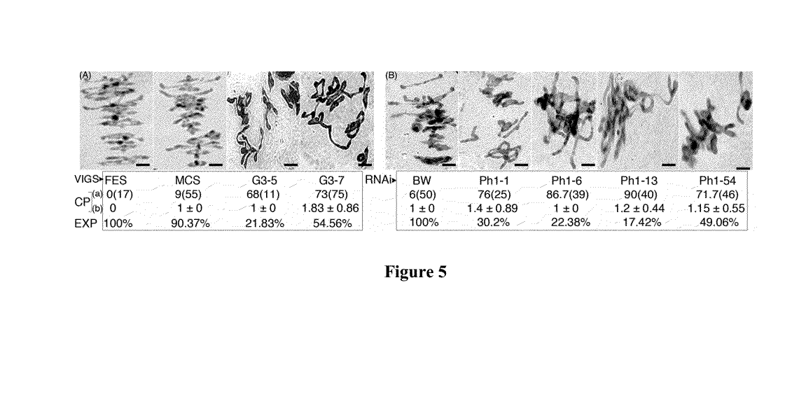

VIGS screening of the 26 candidates identified a gene that was designated as C-Ph1 (candidate Ph1) (LOC_Os9g30320, wheat EST homolog BE498862) (see FIG. 1), the silencing of which showed chromosome pairing behavior characteristic of the Ph1 mutant and deletion lines. Compared to almost all bivalents in 91% of the MI cells of the negative control (VIGS with pγ.MCS, carrying sequence matching multiple cloning site of a plasmid), two of the five plants inoculated with the hairpin construct pγ.C-Ph1hp2 (see FIG. 5; see also Table C below) showed multivalents and higher order pairing in 70.3% of the cells. In addition to multivalents, the MI chromosomes showed severe clustering and disrupted alignment on the MI plate. In comparison, only about 9% of the MI cells of MCS-inoculated plants showed misalignment and multivalents (see FIG. 5).

TABLE C
Chromosomal aberrations in the VIGS and RNAi silenced plants

| Gene | Number of | Average % cells | Average | |
|---|---|---|---|---|
| silencing | silenced | with multivalents/ | bivalents and | |
| method | plants/total | aberration | univalents | Aberration |
| VIGS-Hairpin | 2/5 | 70.35 | 8″ + 0.24′ | Multivalents, clump of |
| (hp) | | | | chromosomes along with |
| | | | | misalignment, some |
| | | | | interlocking |
| VIGS- | 7/20 | 63.3 | 13.46″ + 0.93′ | Multivalents, clump of |
| Antisense (as) | | | | chromosomes along with |
| | | | | misalignment, very few |
| | | | | interlocking, univalents |
| | | | | prevalent in some cells |
| RNAi | 4/7 | 81.1 | 13.68″ + 0′ | Misalignment was very |
| | | | | much prevalent, multivalents |
| | | | | and clumps, interlocking in |
| | | | | all four transgenics |

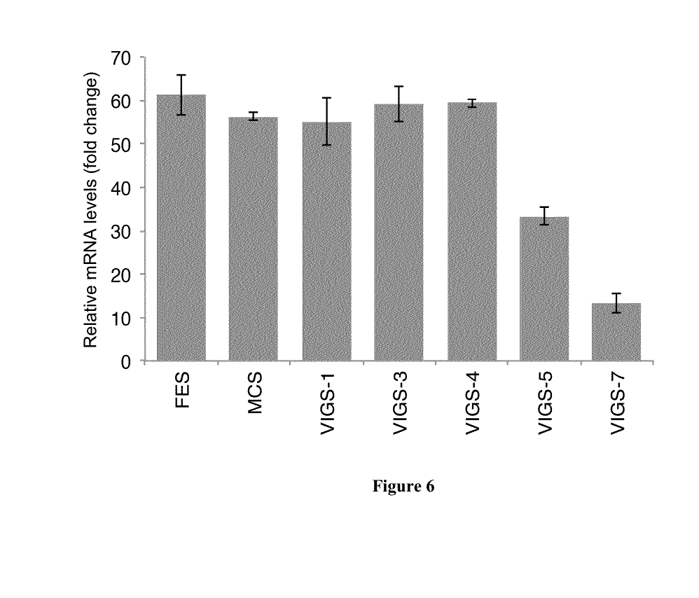

Replicated VIGS experiments with the hairpin and antisense constructs showed a similar phenotype in the silenced plants. The construct pγ.C-Ph1hp1 showed the silencing phenotype in two out of the 20 plants compared to seven out of 20 plants for the pγ.C-Ph1as construct. The two plants with the pγ.C-Ph1hp2 construct showed multivalents and chromosome clustering in 72.7% (plant VIGS-7, see FIG. 5) and 68% (plant VIGS-5, see FIG. 5) of the cells with an average number of only 9.08 and 6.93 bivalents, respectively. Only plant VIGS-7 showed bivalent interlocking in 5% of the cells while no bivalent interlocking was observed in any of the control plants. Measured by quantitative real-time PCR analysis, expression of the gene in plant number VIGS-7 was only 21.83% of the control plants compared to 54.56% in plant number VIGS-5 (see FIG. 6). The expression of the gene in the remaining three plants that did not show any aberration in chromosome pairing, ranged from 89-92% of the control plants (see FIG. 6).

The C-Ph1 gene identified in the present example explains the observations made on the Ph1 gene function. The expression and silencing data clearly suggests that the C-Ph1 gene has multiple functions during meiosis, each controlled by one or more copies of the gene. One of these functions is the initial pairing of both homologues and homoeologues as suggested by the higher expression level of the 5D copy during interphase and the gene silencing phenotype. Chromosome 5B was implicated in the specific function to differentiate homologous from homoeologous chromosome pairing as shown by the unique expression pattern of the 5B copy of the C-Ph1 gene along with the gene silencing phenotype. The 39.7-fold increase in the 5B copy expression between late prophase and MI coincided with the stages when this precise function takes place. The expression of the 5D copy during MI stage suggests additive function of the copy during MI. The 5A copy expressed predominantly during meiosis II suggesting its role in cytokinesis and/or gametophyte development.

In accordance with the interpretation that the 5A and 5D copies of the Ph1 gene share a common function, the predicted proteins of the 5A and the 5D copies of the C-Ph1 gene are very similar except that the 5A copy produces a truncated, perhaps less effective protein. The two proteins share a highly conserved motif corresponding to the exon I that is almost identical among the three homoeologous gene copies, but is absent in the 5B copy proteins. Presence of this highly conserved motif suggests unique function(s) for the two copies including initial pairing of both homologues and homoeologues. Alternatively, the unique function of 5B copy may be due to the lack of this conserved motif along with an insertion of 60 bp that contains an in-frame stop codon thus resulting in smaller proteins. The unique function(s) of the 5B protein(s) may also be due to its very specific expression pattern. The presence of the two 5B copy proteins resulting from alternate splicing suggests multiple functions of the 5B copy. The differences in structure and expression patterns among the three copies of the gene suggest neofunctionalization of the 5B copy with at least one of the functions being different from that of the 5A or the 5D copies. Sequence similarity with diploid species suggests the 5D to be the ancestral copy.

### EXAMPLE 2

### Transient and Stable Silencing of the C-Ph1

Virus-induced gene silencing: The preparation of vector constructs, transcription and inoculation of viral RNAs are generally known to a skilled artisan. On the basis of comparative sequence analysis, the unique gene region for the C-Ph1 homoeologue on chromosome 5B and Cdc2-4 gene was selected for silencing.

FES buffer (abrasive agent used for inoculation) was used as a negative control and the plasmid pγ.MCS (contain 121-bp antisense fragment of the multiple cloning site (MCS) from pBluescript K/S (Stratagene) was used as a ‘virus only’ control in order to differentiate effect of the target gene from that of the virus. For the experiment using an antisense construct, 10 plants were inoculated with pγ.MCS and four plants with FES. Four CS plants were also used as a control. Similarly, for VIGS using pγ.C-Ph1hp2 construct, one and three plants were inoculated with FES and pγ.MCS, respectively. Likewise, for Cdc2-4 gene, five plants each were inoculated with pγ.MCS and FES. To target the gene in PMCs, the flag leaf of the main tiller was inoculated at the boot stage by rubbing. Inoculated plants were lightly misted with water and covered with plastic bags for 16 to 18 hours.

Tissue Collection for Expression Analysis:

Cultivar CS was used for expression analysis from various developmental stages, the tissue was collected as follows: Root tissue was collected from the 10-day old seedlings grown on germination paper in the lab in light; leaf tissue was collected from plants at the Feekes stage 3; the flag leaf and the MI tissue was collected from 3-5 cm spike at the Feekes stage 10.1 and the flag leaf and the spike were individually collected. Tissue for the MII stage was collected by harvesting 6-8 cm spikes at the Feekes stage 10.5-11. The tissue for anthesis (A) stage was harvested as soon as the anthesis started and the five days post-anthesis (5 DPA) was collected five days after the ‘A’ stage.

For sub-staged meiotic tissue, approximately 3-5 cm long spikes that contained meiotically dividing cells, were harvested. One anther from each floret was used for meiotic analysis and the other two were ‘snap frozen’ in liquid nitrogen for subsequent expression studies. This process was continued until all meiotic stages were captured.

Single-Strand Conformation Polymorphism (SSCP Analysis):

Briefly, 2 μg of DNAase treated high quality RNA was converted to first strand cDNA and was diluted to 100 μl with water. One μl of the first strand cDNA was used for the PCR reactions performed with Advantage® PCR Kits Polymerase mixes (Clontech, Catalog #639101), in the presence of 0.2 μl of S³⁵ dATP (Perkin Elmer NEG/033H 1mCi) and 1 pmol/μl of the forward and reverse primers each in a total volume of 10 μl. The PCR product was mixed with an equal volume of a sequencing gel loading buffer containing 95% formamide, 20 mM EDTA, 10 mM NaOH, 0.05% bromophenol blue and 0.05% xylene cyanol. About 5 μl of this mixture was loaded onto 0.4 mm thick 8% polyacrylamide gels. The gels were prepared and run in 0.5×TBE buffer at pH 8.3. For standard runs, the gels were pre-run at a 33 mA constant current for 30-45 mins before running the sample-containing gels at 70 W constant power for 4 hours. For SSCP runs, the gels and the buffer were pre-chilled at 4° C. for at least 5-6 hrs before running it at 10 W for 12-13 hrs at 4° C. An X-ray film was placed on the gels dried using Biorad gel drier, and was exposed for three to seven days. Each sample was size separated both on the standard as well as SSCP gels.

Cytology in Wheat:

The whole wheat inflorescences were harvested and fixed using Carnoy's solution (60 ml ethanol: 30 ml chloroform: 10 ml acetic acid) for several hours at 4° C. From the fixed inflorescence, anther squashes were prepared by aceto-carmine staining. One anther from central floret was squashed in a drop of acetocarmine solution. The debris such as anther walls were removed and the remaining anther was covered with a cover slip. The slide was then heated on a flame briefly followed by slight pressing between a layer of paper towels. This squashing process flattens cell nuclei and spreads out the chromosomes. The slides were first observed under the 10× lens. Once meiotic stage was identified, the cells were then observed under 100× lens. Stained and labeled sections were visualized using Carl-Zeiss AX10 microscope, with images recorded using a axio vision MRm CCD camera and axio vision rel. 4.6.3 software (software imaging system).

Cytology in Arabidopsis:

Whole inflorescences were harvested and fixed using paraformaldehyde (4%) in 1×PBS (phosphate buffered saline). Fixed anthers were stored in 1× buffer A at 4° C. For FISH (fluorescent in-situ hybridization), 5-6 flowers per inflorescence were selected for meiotic analysis based on bud size. Arabidopsis centromeres were visualized by using a cyanine 5-labelled oilgonucleotide YGGTTGCGGTTTAAGTTC (SEQ ID NO:8) (Proligo), which binds to the AL1 repeat present in centromeres. Chromosomes were stained with DAPI (4′,6-diamidino-2-phenylindole). Cells were visualized with Deltavision deconvolution microscope system. 3D images of the entire nuclei were taken along the entire z-stack. Col-8 was used as a wild type control for chromosome pairing analysis.

RNAi Genetic Transformation:

For RNAi-based silencing of the Arabidopsis ortholog, a 200 bp wheat RNAi construct was cloned in the pANDA35HK vector, driven by 35S promoter, and carrying a gene for hygromycin resistance. The Arabidopsis thaliana cv. Col-8 plants were transformed with the construct using the flower dip method (19). Seeds were harvested and planted on nutrient medium containing 15 μg ml⁻¹ hygromycin. Nine plants were selected and subjected to a second round of selection on 15 μg ml hygromycin B. The resistant transgenics were used for cytology.

The RNAi construct for the stable wheat transformation was developed by amplifying 200 bp target sequence from the C-Ph1 gene copy using primers lattbF and lattbR (see Table D below), and cloning into pDONR201 vector using the Gateway cloning system. The confirmed Entry Clone was then transferred to hairpinRNAi Destination vector pHellsgate 8 using LR reaction as described in Kawahara Y et al. (Improvement of the Oryza sativa Nipponbare reference genome using next generation sequence and optical map data, Rice, 2013; 6:1-10). Identity of the clones was first confirmed by restriction-digestion analysis followed by DNA sequencing using Eurofins MWG Operon Simple-Seq services (www.operon.com/fishersci). Sequence verified clone was transferred to Agrobacterium strain C58C1 by electroporation and used in inoculating cultured immature embryos of wheat cultivar Bobwhite. About 800 immature embryos were inoculated with the Agrobacterium strain carrying the RNAi construct. Out of the 800 inoculated embryos, 96 regenerated as plants and 54 were confirmed to be transgenics using various vector specific PCR primers. Seven of the 54 confirmed T₀ transgenic plants were randomly selected for meiotic chromosome pairing analysis.

TABLE D
Gene specific primer sequences used for cloning
the gene homoeologue, real-time quantitative PCR
and SSCP analysis.

| Primer | |
|---|---|
| Name | Sequence 5′ to 3′ |
| 1attbF | GGGGACAAGTTTGTACAAAAAAGCAGGCTCGTCCTACTAAA |
| | CCG (SEQ ID NO: 9) |
| 1attbR | GGGGACCACTTTGTACAAGAAAGCTGGGTACAGGACGAAAC |
| | TGG (SEQ ID NO: 10) |
| CS2F | GGGGACAAGTTTGTACAAAAAAGCAGGCTCGATGGCGCGCC |
| | TCCTCGTTC (SEQ ID NO: 11) |
| CS2R | GGGGACCACTTTGTACAAGAAAGCTGGGTGTTGGCGGCGGG |
| | ACTCTTC (SEQ ID NO: 12) |
| CS8R | GGGGACCACTTTGTACAAGAAAGCTGGGTTACCCATAGACA |
| | CGGGTTCACCATATG (SEQ ID NO: 13) |
| CS9R | GGGGACCACTTTGTACAAGAAAGCTGGGTGCTAGCCTTCAA |
| | AGTGGTGGTTTCATGC (SEQ ID NO: 14) |
| Act-F | ATGTGCTTGATTCTGGTGATGGTGTG (SEQ ID NO: 15) |
| Act-R | CGATTTCCCGCTCAGCAGTTGT (SEQ ID NO: 16) |
| 1-F | CGTCCTACTAAACCG (SEQ ID NO: 17) |
| 1-R | ACAGGACGAAACTGG (SEQ ID NO: 18) |
| G3-F | CGACTACGATGACGCCTTGC (SEQ ID NO: 19) |
| G3-R | GAAGGGGCCGTGTACGGGTGCCGC (SEQ ID NO: 20) |

Bold letters in the primer sequences represent target specific sequence and first 25-27 bases in normal letters represent attB overhangs.

Stable RNAi silencing of the C-Ph1 gene was accomplished by transforming wheat cultivar BW with hairpin RNAi construct pHellsgate8 1-1 involving 200 bp of the gene.

Cloning Full-Length Gene Copies:

The CS2F primer was used as a common forward primer to amplify all three genomic copies. The primers CS2F and CS8R amplified the 5B specific copy of 1014 bp, CS2F and CS9R the 5A-specific copy of 630 bp, and the primer combination CS2F and CS2R amplified the 5D-specific copy of 943 bp. The cDNA copies of the gene were cloned from mRNA isolated from the 3-5 cm spikes at the Feekes scale 10.1. Amplified products were cloned using Gateway vector pDONR201 as per manufacturer's instruction (Invitrogen, CA, USA). Multiple clones were sequenced using Eurofins MWG Operon Simple-Sequence services and data analysis was done using Vector NTI software (Invitrogen, CA, USA).

PCR Reaction Conditions for Cloning the C-Ph1 Gene Homeologues:

The PCR reaction (25 μl) was composed of 100 ng genomic DNA or 50 ng cDNA, 200 mM of each dNTP, 100 nM each primer, 2% DMSO (dimethyl sulfoxide), 1×PCR buffer (Catalog # B9014S, New England Biolabs Inc. MA, USA) and 1 U of DNA polymerase. PCR conditions were 95° C./4 min for initial denaturation, 4 cycles (95° C./1 min, 62° C./1 min, 72° C./1 min) followed by 35 cycles (95° C./1 min, 58° C./1 min, 72° C./1 min), with final extension at 72°/10 min. The PCR fragments were purified from gel by gel extraction kit (NucleoSpin® Gel and PCR Clean-up, Macherey-Nagel Inc, PA, USA) as per the manufacturer's instructions, cloned in pDONR 201 vector (Invitrogen, CA, USA), and sequenced.

PCR Reaction Conditions for Cdc2-4 Amplification:

The PCR reaction (25 μl) was composed of 100 ng Genomic DNA or 5 ng cDNA, 200 mM of each dNTP, 100 nM each primer, 2% DMSO, 1×PCR buffer (Catalog # B9014S, New England Biolabs Inc. MA, USA) and 1 U of DNA polymerase. PCR conditions were 95° C./4 min for initial denaturation, 4 cycles (95° C./1 min, 62° C./1 min, 72° C./1 min) followed by 35 cycles (95° C./1 min, 58° C./1 min, 72° C./1 min), with final extension at 72°/10 min. The PCR products of Cdc2-4 specific primers were resolved on Roche LightCycler® 480 (Roche Diagnostics, USA) using Melt Curve analysis and on 2% agarose gel. The primer sequences for the sequence tagged site (STS) primers for wheat Cdc2-4 gene were kindly provided by Dr. Graham Moore, JIC.

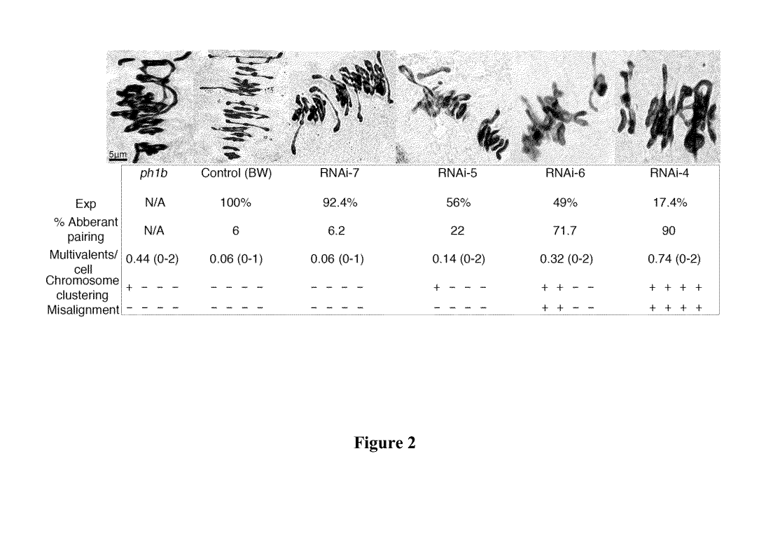

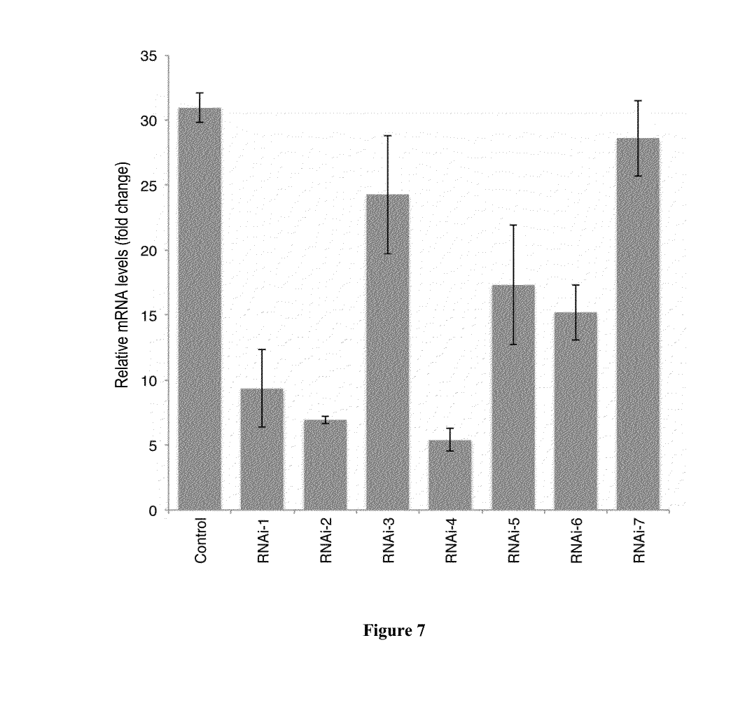

Compared to the negative control (BW), reduction in the gene expression among these seven plants ranged from 7 to 83% (see FIG. 7). Transcriptional suppression between 22 to 44% appears to be necessary to induce homoeologous chromosome pairing. Two of the seven plants (RNAi-7, RNAi-3) that respectively exhibited 7 and 22% transcript suppression, showed relatively normal chromosome pairing with only 6.2% and 14.2% of the MI cells, respectively showing aberrant chromosome pairing. RNAi-3 showed slight although non-significant multivalents along with misalignment. Multivalents were not observed in RNAi-7 although slight misalignment was observed similar to that of the negative control that showed misalignment in only 6% of the cells (see FIG. 2 and FIG. 5). These two plants were fully fertile.

RNAi-5 with 44% reduction in gene expression was the most interesting as it showed chromosome pairing phenotype similar to that seen in the ph1b mutant (see FIG. 2 and FIG. 5). Multivalents were observed in 22% of the cells (see FIG. 2). The number of bivalents in this plant were 12 compared to 16 in the ph1b mutant (see Table B above).

TABLE E
Chromosome pairing abnormalities in the wild type and the Arabidopsis
silenced plants

| | | Silenced plants | | Wild-type | |
|---|---|---|---|---|---|
| | Pairing | | Cells with | | Cells with |
| Meiotic | Phenotype | Cells | abnormal | Cells | abnormal |
| stage | analyzed | analyzed | pairing | analyzed | pairing |
| Leptotene | Centromere | 15 | 15 | 8 | 0 |
| | coupling | | | | |
| Zygotene | Multivalent | 20 | 19 | 10 | 0 |
| | formation | | | | |
| Pachytene | Multivalent | 20 | 18 | 10 | 0 |
| | formation | | | | |

As is the case for the ph1 mutants, RNAi-5 was partially fertile. Misalignment and chromosome clustering was not commonly seen in this plant. Reduction in gene expression in the remaining four plants ranged from 50 to 83% (see Table C). The number of bivalents in these four plants ranged from 10.6 to 14.3 compared to 19.7 in the negative control. The number of MI cells showing multivalents, chromosome clustering and misalignment ranged from 71.7 to 90% (see FIG. 5).

The plant (RNAi-6) showing 50% reduction in gene expression exhibited a more severe phenotype than RNAi-5 with additional chromosome clumping and disrupted alignment on the MI plate. This plant exhibited aberrant chromosome pairing in 71.7% of the cells (see Table C). There was a significant increase in the levels of chromosome clumping and misalignment along the MI plate with the further 31.64% reduction in gene expression. RNAi-4 showed the maximum level of silencing (see FIGS. 2 and 5). Bivalent interlocking, which was not observed in the negative control, was present in 31.2% of the MI cells of these four transgenic plants. Overall, chromosome pairing aberration was very severe in these four plants compared to that seen in the ph1b mutant (see FIGS. 2 and 5). These four plants were completely sterile but exhibited no other phenotypic abnormality.

VIGS and RNAi silenced plants showed the chromosome pairing aberrations observed in the Ph1 gene mutations along with some additional phenotypes including chromosome clumping and disrupted alignment on the MI plate. Both in the VIGS as well as in the RNAi plants, the severity of the chromosome pairing phenotype correlated well with the level of gene silencing. One of the RNAi plants (RNAi-5) with 44% reduction in the gene expression showed chromosome pairing phenotype similar to that seen in the ph1 mutants and the lines lacking the Ph1 gene (see FIG. 2). Characteristic of the ph1 mutants, multivalents were observed in this plant without any disruption in chromosome alignment on the MI plate or chromosome clumping (see FIG. 2). These results suggest that about 44% reduction in expression of the gene can demonstrate the aberrant chromosome pairing phenotype similar to that observed in the Ph1 gene mutants. Previously, bivalent interlocking was observed in ph1b mutants as well as in the plants lacking the 5B or the 5D copies, and also in tri-isosomic 5BL plants. Bivalent interlocking in the C-Ph1 silenced plants was also observed. The bivalent interlocking is probably caused by pairing between distant homologues in the absence of the gene. Bivalent interlocking in the plants carrying triple dose of chromosome 5BL is probably due to the dosage effect of Ph1 on the relative separation of homologues prior to meiosis. This could also be due to silencing of the gene triggered by the higher copy number.

RNAi and VIGS plants with a gene silencing of more than 44% resulted in chromosome clustering and misalignment of chromosomes on the MI plate in addition to the expected multivalent formation. This phenotype was not observed in any of the NT lines probably because multiple copies of the gene perform the same function and loss of a copy in NT lines is compensated for by the other copies.

The data presented herein suggest a plausible explanation of the above mentioned observations. Firstly, expression of the 5B copy increases between late prophase and MI coinciding with the stages when centromere-microtubule interactions take place. Secondly, transient VIGS as well as stable RNAi silencing of the C-Ph1 gene resulted in severe centromere clustering along with disrupted alignment of chromosomes on the MI plate, suggesting a plausible role in centromere-microtubule interaction. This dramatic clustering and misalignment was not observed in the absence of either one of the three gene copies during previous studies suggesting that two or more of the gene copies act in an additive manner to accomplish this very important function. Also, expression pattern of the 5B copy of the C-Ph1 gene closely coincides with that of the motor protein CENP-E, a kinetochore-associated protein involved in the sustained movement of chromosomes leading to proper alignment on the MI plate. Taken together, these and the observations of disrupted chromosome alignment on the MI plate in the VIGS and the RNAi plants, suggest that either the C-Ph1 gene functions by regulating centromere-microtubule interaction as was previously suggested, or this is one of the additional functions of the gene where two or more copies of the gene have the same function which is in addition to regulating HECP.

Studied in the root-tip cells, chromosomes in ph1b mutant showed higher mitotic association of homoeologues and hypersensitivity to colchicine as compared to that in the normal CS; and disrupted centromere-microtubule association was suggested to be the cause. A low level of expression of the 5B copy of the C-Ph1 gene was observed in all mitotically dividing tissue including roots and leaves, suspecting its role in mitotic cell division. This also supports the previous reports that Ph1 gene functions during mitotic and particularly premeiotic stages affecting chromosomal movement towards the poles and consequently, their arrangement in the nucleus. This may effect premeiotic association of homologous chromosomes and relative separation of homoeologues thus determining exclusive homologous pairing in wheat already before the commencement of synapsis.

### EXAMPLE 3

### Structure of the C-Ph1 Gene

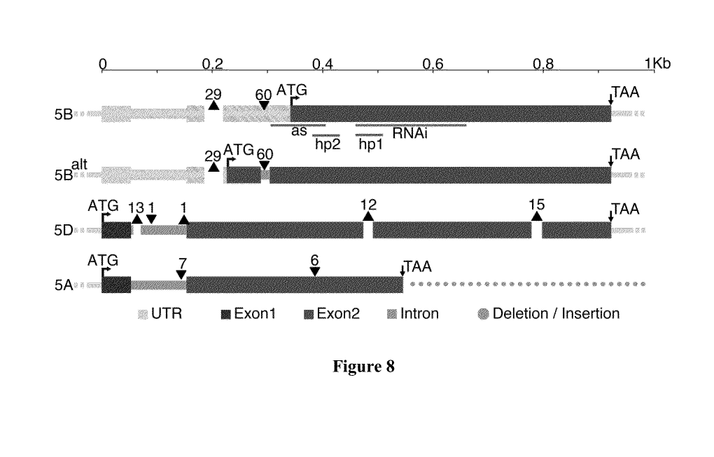

Detailed bioinformatics and sequence analyses revealed three genomic and cDNA copies of the gene, one on each of the three wheat group 5 chromosomes (see FIG. 8). At DNA level, the three homoeologues from cv. CS were 90% similar and the differences were due to several structural changes including deletions and insertions (see FIG. 8). The 5B copy of the gene sequence showed two novel insertions of 46 bp and 14 bp (referred to as 60 bp insertion) in the exon II (see FIG. 8). A deletion of 29 bp was observed 80 bp upstream of the two insertions. There were two 5D specific deletions of 12 bp and 15 bp present in the exon II. Additional differences among the homoeologues were a 13 bp deletion in the 5D copy present 11 bp downstream of the exon-intron junction; and two insertions in the 5A copy: a 7 bp in the intron and 6 bp in the exon II (see FIG. 8). The 5′ UTR (untranslated region) of the 5B copy was 96.8% similar to that of the 5D copy and 94.3% to the 5A copy. Similar comparison between the 5A and 5D copies showed 92.6% similarity. The 3′ UTRs of the 5B and 5D copies are 92.6% similar and the differences were mainly due to an 11 bp insertion in the 5B copy along with six homoeologous sequence variants (HSVs). The 3′ UTR of the 5A copy did not match with either the 5B or the 5D copies mainly due to a major deletion/insertion. Of the HSVs among the three gene copies, 26.7% were synonymous and 73.3% were non-synonymous. Overall, percentage of non-synonymous bases in the gene was 77.8% with the remaining 22.3% being synonymous.

The genomic copy of 5B was the largest (954 bp) compared to the 5D (883 bp) and the 5A copies (539 bp). Excluding insertions and deletions, the 5B genomic copy is 95% similar to that of the 5D copy. The similar number is 94.4% for the comparison between 5B and 5A and 85.9% for the comparison between 5A and 5D. Overall, the genomic copies of the three homoeologues shared 41% DNA sequence similarity.

Among the three gene copies, 5A produced the smallest transcript (excluding 5′ and 3′ UTR) of 420 bp by splicing an intron of 120 bp while the 5D produced a transcript of 783 bp by splicing an intron of 100 bp (see FIG. 8). The 5B copy of the gene showed signs of alternate splicing to produce transcripts of 954 bp and 763 bp. The difference between the two variants was two introns of 113 bp and 78 bp, which were spliced to generate the 763 bp version and were retained to generate what turned out to be the largest transcript among all gene copies (see FIG. 8). The 5B copy with the larger transcript generated a smaller protein than its alternate form due to retention of the intron around the 60 bp addition that contained an in-frame stop codon (see FIG. 8). Predicted proteins from either of the two splice variants from the 5B were smaller than that produced by the 5D copy but was larger than that of the 5A copy.

Predicted proteins from the 5B copy of the gene were of 204aa (amino acids) and 221aa in length compared to 174aa from the 5A and 260aa from the 5D copy. The most conserved part of the gene corresponding to the exon I was not present in either of the predicted proteins from the 5B copy but was present in the predicted proteins from the 5A and the 5D copies. The two proteins representing the 5B copy of the gene were 91% similar with the only difference being additional 17aa on the N terminus in the larger version. Not counting the 86aa deletion in 5A created by a premature stop codon, the predicted proteins of the three copies were 82% similar. Considering all deletions and insertions however, the 5A copy protein is only 25-30% and 46.4% similar to that of the 5B and 5D copy proteins, respectively. Similarly, 5D and the 5B copy proteins were 68 to 74% similar.

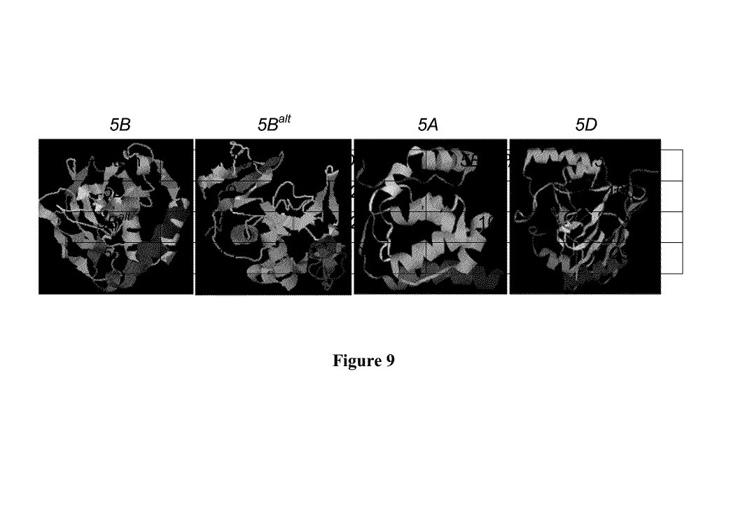

Likewise, the predicted 3D structures of the four proteins were significantly different, indicating functional divergence of the homoeologues after allopolyploidization (see FIG. 9). Surprisingly, the 3D structure of the two alternate splice variants of the 5B copy showed only 43% similarity. The 3D structure of either of the two 5B forms showed only 27-29% similarity with that of 5A or the 5D copies (see FIG. 9). The similarity between the 3D structure of the 5A and the 5D proteins was only 13%. These structural, functional, and expression differences suggest that various protein forms of the gene may have different functions during meiosis, analogous to the previous observations on the Pairing homeologous gene 1 (Ph1) gene, implicating it in multiple meiotic processes.

### EXAMPLE 4

### C-Ph1 Gene Expression Pattern

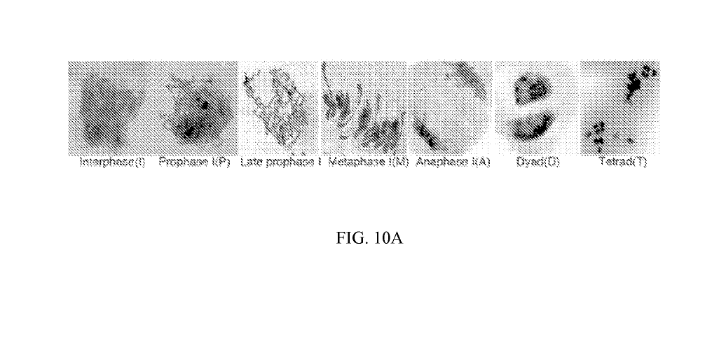

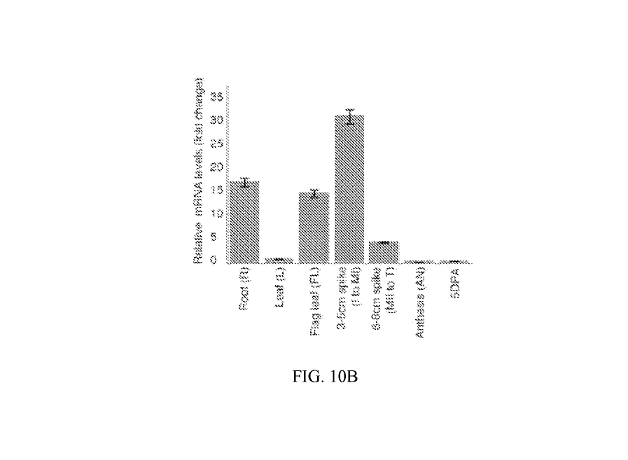

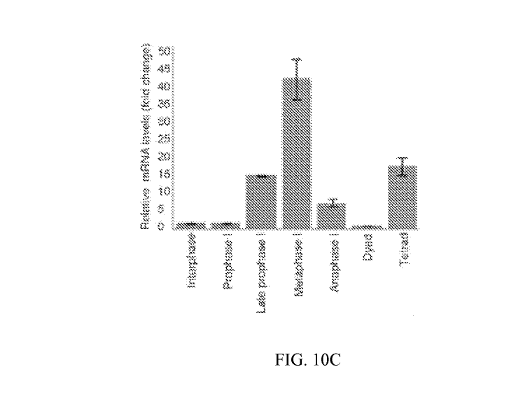

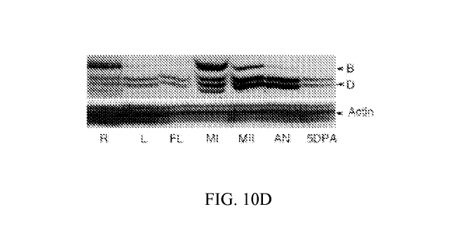

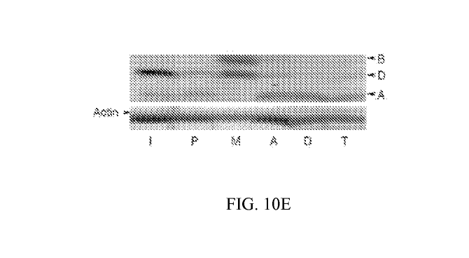

Quantitative real-time expression analysis representing cumulative expression of all homoeologues showed that the gene primarily expresses during the post-flower initiation stages although significant expression was observed in the roots as well (see FIGS. 10A-10E). The maximum expression of the gene was, however, during meiosis I. Flag leaf of the plants undergoing meiosis also showed significant expression. Essentially no expression was observed in mature pollen grains or the subsequent seed development stages.

Plant Material.

Plant material used in this example included wild-type hexaploid wheat (T. aestivum cv. Chinese Spring and cv. Bob White); a Chinese Spring mutant lacking the Ph1 locus (ph1b); wheat homoeologous group 5 NT lines, and a series of 5BL deletion lines. Based on the efficient utilization of the cv. CS for chromosome squash preparations, it was selected as an ideal cultivar for VIGS. CS, NT and deletion lines were used for mapping and cloning experiments. Bobwhite was used for RNAi experiments as it can be efficiently transformed using the Agrobacterium-mediated gene transfer.

To ‘pinpoint’ the gene expression pattern during various meiotic stages, one of the three anthers from each floret was used for the meiotic chromosome analysis and the remaining two were used for the real-time gene expression analyses.). All three anthers from a single wheat floret are known to be at the developmentally identical stage. Chromosome spreads of pollen mother cells (PMCs) from wheat cv. Chinese Spring denoting the various stages of meiosis are shown in FIG. 10A. Expression of the gene increased 13-fold in transition from prophase I to late prophase I, followed by further increase of about 26-fold during MI (FIG. 10C). Relative to MI, the expression dropped by 34-fold during anaphase I followed by further drop of 6.4-fold during the dyad stage (FIG. 10C). Maximum expression of the gene was observed during MI. Surprisingly, there was 16.5-fold increase in gene expression during the tetrad stage suggesting additional functions of the gene.

Expression analysis of each of the gene copy individually by single-strand conformation polymorphism (SSCP) analysis revealed that the three copies of the gene have dramatically different expression patterns. With the exception of roots where almost all copies showed expression, the 5B copy specifically expressed in 3-5 cm long spike, which in CS contains meiotically dividing cells (FIG. 10D). Expression of the 5B copy dropped significantly in the 6-8 cm spike that contains cells at meiosis II stage. Essentially no expression was observed for the copy at the mature anther stage (FIG. 10D). Expression pattern of the 5D copy was very different from that of the 5B copy. Unlike 5B, the 5D copy showed low-level of expression in the leaves and the 3-5 cm spike, but none during anthesis or five days post-anthesis (SDPA) (FIG. 10D). The 5A copy expressed predominantly during meiosis II and showed very little expression in the 3-5 cm spike suggesting its role in cytokinesis and/or gametophyte development.

In the sub-staged meiotic anthers, the 5B copy specifically expressed during MI and a low level expression was also seen during anaphase I. No expression was observed for the copy in the subsequent meiotic stages (FIG. 10E). Unlike 5B, the 5A copy expressed during anaphase I, dyad and tetrad stages. Compared to the other two, the 5D copy showed significantly higher expression during interphase. A significant amount of expression of the 5D copy was also observed during prophase I and MI (FIG. 10E).

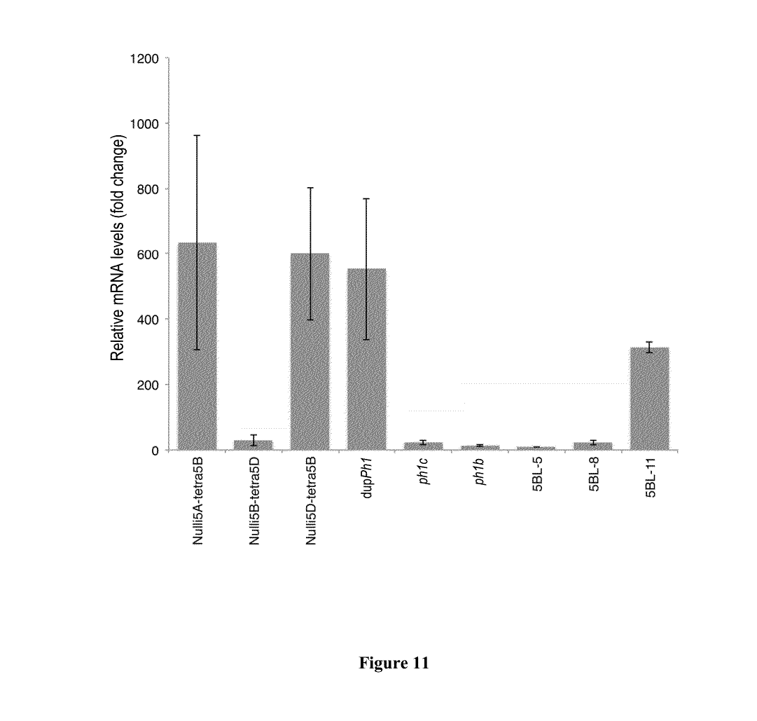

Additionally, the expression of 5B copy was also analyzed in the wheat homoeologous group 5 nullisomic-tetrasomic (NT) lines, ph1 mutants and a series of 5BL deletion lines during meiosis (see FIG. 11). The 5B-specific transcript was absent in the Nulli5B-TetraSD line as compared to that in the NulliSA-TetraSB and NulliSD-TetraSB lines (see FIG. 9). Essentially no expression was observed in the ph1b and ph1c mutant lines in contrast to the expression levels in the line carrying duplication (dupPh1). A significant amount of expression was observed in 5BL-11 (fraction length, FL-0.59) while 5BL-5 (FL-0.54) and 5BL-8 (FL-0.52) essentially showed no expression. These results provide additional line of evidence to further confirm that the newly identified C-Ph1 gene in this study corresponds to the Ph1 locus.

### EXAMPLE 5

### Gene Orthologues in Other Plants

Structurally conserved orthologues of the C-Ph1 gene were observed in all studied monocots including rice, barley, maize, and Brachypodium. The rice orthologue (LOC_Os09g30320) maps on Chromosome 9, the Brachypodium orthologue (Bradi4g33300) on chromosome 4, and the maize orthologue (GRMZM2G078779) on chromosome 7. The gene showed a typical pattern of three exons and two introns in all the species tested except for maize, which had two additional splice variants. Besides the conserved BURP domain (named based on four typical members, BNM2, USP, RD22, and PG1β), a 45 bp first exon followed by a 94-138 bp intron and a 114-138 bp second exon were observed. The size of second intron in rice was 599 bp, which is comparatively larger than 97, 119 and 122 bp in maize, Brachypodium and barley, respectively. The third exon was between 596-644 bp in size, contains BURP domain related sequences. The transcribed part of the barley copy showed 90% similarity with the 5B copy of the C-Ph1 gene, while the other species had 71-72% similarity. Structurally, the putative C-Ph1 orthologue from several diploids including barley, maize, Brachypodium, and rice resembled more with the 5D copy suggesting this likely represent the conserved and ancestral version of this gene.

Using studies with the available microarray based expression data, the maize orthologue of the C-Ph1 gene showed the highest expression in the meiotic tassel and anthers containing the pollen mother cell meiotic stages. The developing endosperm and kernel also showed significant level of expression but no other developmental stage showed any expression of the gene. Similarly, the rice orthologue of the gene, LOC_Os09g30320, also showed the highest expression in the heading panicle and stamens containing meiotic tissues and no expression was observed in any other developmental stages. The barley orthologue was identified from ESTs derived from immature male inflorescences although a detailed expression pattern of the gene in barley is not yet known.

Initially, DNA sequence analysis and domain/motif search identified At5g25610 as the putative Arabidopsis orthologue of Os09g30320. At5g25610 encoded a BURP domain containing protein with a putative function in dehydration stress response as it encodes dehydration-induced protein RD22 thus making it a less likely candidate in Arabidopsis. Sequence comparison at the DNA or protein level identified an orthologue of the wheat gene in Arabidopsis although with poor conservation. At1g78100 mRNA matched perfectly with the 22nt of wheat RNAi fragment. The gene expresses during anthesis and various other developmental stages thus making it a likely candidate for gene orthologue. Poor sequence conservation among orthologues has been well documented for many other meiotic genes.

### EXAMPLE 6

### Gene Function in Arabidopsis

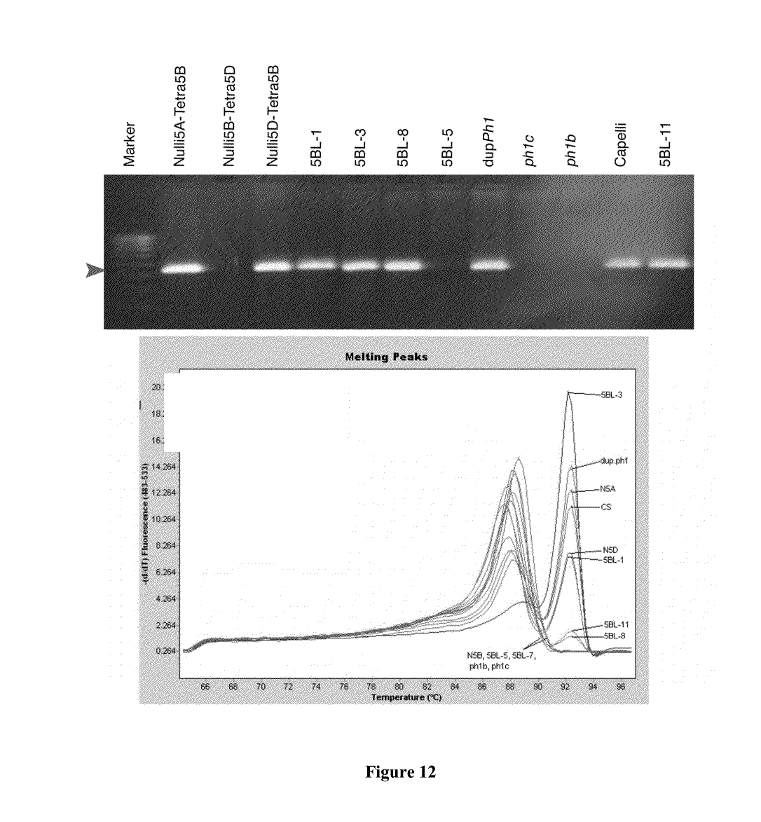

With the assumption of functional conservation of the gene between Arabidopsis and wheat due to conserved catalytic motifs, we performed RNAi-based silencing of the Arabidopsis orthologue. The resistant transgenic lines along with wild-type Col-8 were analyzed for meiotic chromosome pairing analysis and the results are shown in FIG. 12 and summarized in Table E and Table F.

TABLE E
Chromosome pairing abnormalities in the wild type and the Arabidopsis
silenced plants

| | | Silenced plants | | Wild-type | |
|---|---|---|---|---|---|
| | Pairing | | Cells with | | Cells with |
| Meiotic | Phenotype | Cells | abnormal | Cells | abnormal |
| stage | analyzed | analyzed | pairing | analyzed | pairing |
| Leptotene | Centromere | 15 | 15 | 8 | 0 |
| | coupling | | | | |
| Zygotene | Multivalent | 20 | 19 | 10 | 0 |
| | formation | | | | |
| Pachytene | Multivalent | 20 | 18 | 10 | 0 |
| | formation | | | | |

Twenty-five cells were imaged and analyzed for chromosome pairing defects during early stages of leptotene to pachytene. In all of the cells analyzed, the wild-type Arabidopsis showed 5 bivalents (FIG. 4C) in contrast to average 3.05 in the silenced plants. On an average 0.9 quadrivalents and 0.05 hexavalents per meiotic cell were observed in the RNAi plants whereas no quadrivalent or hexavalent was observed in the wild-type plants (see Table F). The RNAi plants showed multiple associations in the form of centromere coupling in all of the analyzed cells at the leptotene stage (see FIG. 5 and Table E) while no such centromere coupling was observed in the wild-type. The centromere coupling lead to the formation of multivalents during zygotene and pachytene stages. The silenced plants showed multivalent formation in 90-95% of the analyzed cells in the zygotene and pachytene stages (see FIG. 5 and Table E). The wild-type was normal and showed no such chromosomal aberrations.

TABLE F
Chromosome pairing analysis during zygotene in the wild type and the Arabidopsis
silenced plants

| | Number | Average | Average | Average | Average |
|---|---|---|---|---|---|
| | of cells | number of | number of | number of | number of |
| Plant type | analyzed | univalents/cell | bivalents/cell | quadrivalents/cell | hexavalents/cell |
| Wild-type | 10 | 0 | 5 | 0 | 0 |
| Silenced | 25 | 0 | 3.05 | 0.9 | 0.05 |
| Plants | | | | | |

Silencing of the gene in Arabidopsis also showed a phenotype similar to that of wheat. In particular, severe chromosome clustering was observed, especially of the centromeres first seen at leptotene stage in the form of centromere coupling, which later lead to the formation of multivalents during zygotene and pachytene stages. Arabidopsis thaliana is believed to be an ancient polyploid with ancestral genome represented as segmental duplications. The multivalents observed in the C-Ph1 silenced plants is probably due to pairing of the duplicated segments in non-homologous chromosomes. These results suggest that the gene is functionally conserved between wheat and Arabidopsis although the sequence level conservation appears only for the catalytic motifs rather than the entire gene. Poor sequence conservation among orthologues has been well documented for many other meiotic genes. Sporulation protein (SPO11), despite being functionally conserved from yeast to humans for its role in double-strand break (DSB) formation, shares only 23% protein sequence similarity between yeast and mice.

Besides Spo11, identification of orthologues for other genes involved in meiosis such as meiosis-specific 4 (Mei4) and meiotic-recombination 114 (Rec114) has also been very difficult. These genes, despite their functional conservation, share only 6 to 8% of the protein sequence similarity between budding yeast and mice. The meiotic genes, however, show a strong functional conservation across eukaryotes probably due to conservation of catalytic motifs. The identified gene therefore follows the same level of sequence conservation among eukaryotes as that was observed for many other meiotic genes.

### EXAMPLE 7

### Cdc2-4 May not be a Good Candidate for the Ph1 Gene

Cell division cycle 2 (Cdc2-4), a cell cycle regulator gene encoding cyclin dependent protein kinase was reported to be a candidate for the Ph1 gene action (1). Cdc2 related genes are known to affect chromosome condensation (2). The Cdc2-4 gene is present in chromosome deletion lines 5BL-1, 5BL-3, 5BL-8 (see FIG. 12) that are known to lack the Ph1 gene as their chromosome pairing matched with that of the ph1b and other Ph1 gene mutants. Expression of Cdc2-4 was the same between CS and the mutant and deletion lines especially during meiosis.

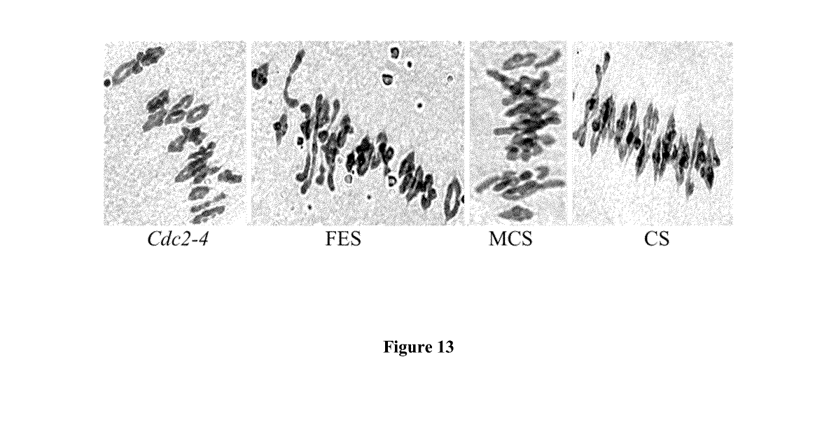

VIGS analysis with a 96 bp antisense construct targeting Cdc2-4 gene showed normal chromosome pairing at meiotic MI (see FIG. 13 and Table G). The VIGS plants showed an average of 20.9 bivalents as compared to 21 in the MCS and FES control plants (see FIG. 13).

TABLE G
Chromosome pairing analysis of Cdc2-4 VIGS inoculated plants.
Chromosome pairing at metaphase I of inoculated (pγ.Cdc2-4as
and pγ.MCS) and uninoculated (FES rubbed) plant. Bivalents are
indicated as average values of rod and ring chromosomes. Cell
number indicates the total number of cells analyzed.

| | Univalents | Multivalents | Bivalents (mean) | | Cell |
|---|---|---|---|---|---|
| Plant | (mean) | (%) | Rod | Ring | number |
| Cdc2-4 | 0.2 | 0 | 3.04 | 17.86 | 50 |
| MCS | 0 | 0 | 3.53 | 17.46 | 47 |
| FES | 0 | 0 | 2.3 | 18.68 | 50 |

No multivalents were observed in the MCS and FES inoculated plants or in the non-inoculated CS plants (see FIG. 13). On average, 50 cells were counted for each Cdc2-4 inoculated, MCS and FES plants. Furthermore, when an antisense oligodeoxynucleotides was introduced targeting the cdk-like gene complex using detached tiller method, the chromosome pairing observed at MI appeared normal contrary to the Ph1 mutants (ph1c and ph1b). The abnormality symptoms were pronounced only during the late stages of Meiosis II, which included tetrads with missing nuclei and presence of micronuclei in the microspores.

1. A plant or plant part comprising a homoeologous recombination of chromosomes, wherein the plant or plant part comprises reduced expression of a Ph1 gene.

2. The plant or plant part of claim 1, wherein the plant or plant part is a hybrid plant or plant part.

3. The plant or plant part of claim 1, wherein the plant or plant part is a transgenic plant or plant part.

4. The plant or plant part of claim 1, wherein the plant or plant part is wheat.

5. The plant or plant part of claim 1, wherein the plant or plant part is a RNAi-5 plant or plant part.

6. The plant or plant part of claim 1, wherein the reduced expression of a Ph1 gene is between 22% and 83% reduction in gene expression compared to a negative control.

7. The plant or plant part of claim 1, wherein the Ph1 gene is C-Ph1.

8. The plant or plant part of claim 1, wherein the plant or plant part is inoculated with a vector construct comprising a nucleotide sequence selected from the group consisting of SEQ ID. NO:4, SEQ ID. NO:5, SEQ ID. NO:6, and SEQ ID. NO:7.

9. A method of reducing gene expression of a Ph1 gene in a plant, said method comprising the step of silencing the Ph1 gene in a chromosome of the plant.

10. The method of claim 9, wherein the silencing is virus induced gene silencing (VIGS).

11. The method of claim 9, wherein the plant is a polyploid plant.

12. A vector construct comprising a nucleotide sequence selected from the group consisting of SEQ ID. NO:4, SEQ ID. NO:5, SEQ ID. NO:6, and SEQ ID. NO:7.

13. The vector construct of claim 12, wherein the vector construct is a hairpin construct.

14. The vector construct of claim 12, wherein the vector construct is an antisense construct.

15. The vector construct of claim 12, wherein the nucleotide sequence is selected from the group consisting of SEQ ID. NO:5, SEQ ID. NO:6, and SEQ ID. NO:7.

16. The vector construct of claim 12, wherein the vector construct is pγ.C-Ph1hp1.

17. The vector construct of claim 12, wherein the vector construct is pγ.C-Ph1hp2.

18. The vector construct of claim 12, wherein the vector construct is pγ.C-Ph1as.

19. The vector construct of claim 12, wherein the vector construct is pHellsgate8 1-1

20. The vector construct of claim 12, wherein the nucleotide sequence consists of a sequence selected from the group of SEQ ID. NO:4, SEQ ID. NO:5, SEQ ID. NO:6, and SEQ ID. NO:7.
